(19) United States
(12) Patent Application Publication
(10) Pub. No.: US 2026/0055405 A1
(43) Pub. Date: Feb. 26, 2026

(54) TREATMENT

(71) Applicant: THE UNIVERSITY COURT OF THE UNIVERSITY OF EDINBURGH, Edinburgh (GB)

(72) Inventors: Andrew BAKER, Edinburgh (GB); Serena ZACCHIGNA, Trieste (IT); Mauro GIACCA, London (GB)

(21) Appl. No.: 19/104,460

(22) PCT Filed: Aug. 18, 2023
(86) PCT No.: PCT/GB2023/052170

§ 371 Date: Feb. 18, 2025

(30) Foreign Application Priority Data
Aug. 19, 2022 (GB) 2212125.5

(51) Int. Cl. C12N 15/113

(52) U.S. Cl. CPC C12N 15/113; C12N 2310/141

(57) ABSTRACT

Disclosed is a cohort of anti-proliferative microRNAs (miRs) wherein each member of the cohort (or combinations thereof) represent a target for modulating cell proliferation (and migration), influencing (or modulating) vascular remodelling and the treatment of various vascular complications, vascular injury vascular disease and (for example) disorders, diseases, syndromes and/or conditions which affect vessels and/or vascular systems of the human or animal body.

### FIELD

The present disclosure provides compositions, medicaments and methods for use in treating or preventing vascular complications and/or injury or damage following surgery.

### BACKGROUND

Aberrant proliferation of vascular smooth muscle cells is known to be involved in the acute (e.g. coronary bypass surgery using saphenous veins) and chronic (e.g. development of atherosclerosis) responses to vascular injury.

There is no treatment for late vein graft failure, except for percutaneous coronary interventions or repeating the bypass surgery, which is considered challenging and highly risky. Most effort is therefore focused on prevention (statins and antiplatelets (e.g. aspirin)) but these do not address the problematic principle that early pathological remodelling of the graft post-implantation creates the necessary environment to promote superimposed atherosclerosis; hence, preventing early pathological graft remodelling will lead to improvements in vein graft patency rates in the long term. Several surgical protocols (such as total arterial revascularisation) have also been shown to reduce the rate of vein graft failure, but most are difficult to implement outside of several specialised centres and have associated risks to the patient. If successful, the proposed approach will have a high likelihood of being implemented at a wide scale.

### SUMMARY

The present disclosure is based on the discovery of a cohort of anti-proliferative microRNAs (miRs) wherein each member of the cohort (or combinations thereof) represent a target for modulating cell proliferation (and migration), influencing (or modulating) vascular remodelling and the treatment of various vascular complications, vascular injury vascular disease and (for example) disorders, diseases, syndromes and/or conditions which affect vessels and/or vascular systems of the human or animal body.

Within the context of this disclosure, any of the disclosed miRs may be targeted to, for example, modulate their level of expression in a cell. By way of example, the level of expression of any of the disclosed miRs may be increased or decreased as required. Without wishing to be bound by theory, it has now been shown that the level of expression of any of the disclosed miRs is linked to potentially beneficial therapeutic effect. For example and again without wishing to be bound by theory, it has been found that by targeting one or more of the disclosed miRs, it is possible to modify or modulate vascular smooth muscle cell proliferation. This may help modify blood vessel wall architecture/structure following injury. As such, the disclosure provides a cohort of miRs each member of which may be targeted as a means to modulate (for example inhibit) vascular smooth muscle cell proliferation and/or for the treatment or prevention of (vascular) complications, (vascular) injury and/or (vascular) disease characterised by vascular smooth muscle cell proliferation.

The present disclosure provides compounds and compositions for various therapeutic uses, the use of compounds and compositions for the manufacture of therapeutically effective medicaments and methods of treating a variety of diseases, disorders and conditions.

One particular application of the compounds/compositions, medicaments, uses and methods described herein may be in the treatment or prevention of vein graft failure/late vein graft failure.

Vascular smooth muscle cells (VSMCs) and VSMC-derived cells are a major source of plaque cells and extracellular matrix at all stages of atherosclerosis. As such, the various compounds/compositions, medicaments, uses and methods described herein may be further applied to the treatment or prevention of, for example, diseases or complications associated with vascular plaque formation and/or atherosclerosis.

It should be noted that as used herein, the term VSMC may embrace human saphenous vein SMC (HSVSMC). Moreover, throughout this specification the terms “comprise” and/or “comprising” are used to denote that embodiments “comprise” the noted features and as such, may also include other features. However, in the context of this disclosure, the terms “comprise” and “comprising” encompass embodiments which “consists essentially of”the relevant features or “consists of”the relevant features.

In a first aspect, the disclosure provides a miR modulator for use in:
modulating VSMC proliferation;
treating or preventing diseases or conditions characterised by VSMC proliferation;
treating or preventing vein graft failure/late vein graft failure;
treating or preventing vein graft failure/late vein graft failure following surgery;
treating or preventing vein graft failure/late vein graft failure following coronary;
bypass surgery, including, for example using saphenous veins;
treating or preventing atherosclerosis;
treating, preventing or modulating vascular remodelling;
treating or preventing vascular injury; and/or
treating or preventing vascular injury where VSMC proliferation is an important phenotype.

In a second aspect, the disclosure provides a method of:
modulating VSMC proliferation;
treating or preventing diseases or conditions characterised by VSMC proliferation;
treating or preventing vein graft failure/late vein graft failure;
treating or preventing vein graft failure/late vein graft failure following surgery;
treating or preventing vein graft failure/late vein graft failure following coronary;
bypass surgery, including, for example using saphenous veins;
treating or preventing atherosclerosis;
treating, preventing or modulating vascular remodelling;
treating or preventing vascular injury; and/or
treating or preventing vascular injury where VSMC proliferation is an important phenotype; said method comprising administering a miR modulator to a subject in need thereof. The modulators may be administered in a therapeutically effective or modulatory amount. The subject in need thereof may be a human or animal subject.

In a third aspect, the disclosure provides the use of a miR modulator for the manufacture of a medicament for use in:
modulating VSMC proliferation;
treating or preventing diseases or conditions characterised by VSMC proliferation;
treating or preventing vein graft failure/late vein graft failure;
treating or preventing vein graft failure/late vein graft failure following surgery;
treating or preventing vein graft failure/late vein graft failure following coronary;
bypass surgery, including, for example using saphenous veins;
treating or preventing atherosclerosis;
treating, preventing or modulating vascular remodelling;
treating or preventing vascular injury; and/or
treating or preventing vascular injury where VSMC proliferation is an important phenotype.

It should be noted that the phrase “diseases or conditions characterised by VSMC proliferation” may embrace, for example in stent restenosis, arteriovenous fistulas, intimal thickening (hyperplasia), vessel occlusion and complication arising from constricted/restricted blood flow. As such, the various methods, uses or medicaments of this disclosure may be used to:
treat or prevent in stent restenosis,
treat or prevent arteriovenous fistulas;
treat or prevent intimal thickening (hyperplasia);
treat or prevent vessel occlusion; and/or
treat or prevent complications arising from constricted/restricted blood flow.

Vascular remodelling is often aberrant and therefore one may exploit the uses, methods and medicaments described herein as a means to inhibit or prevent certain vascular remodelling events/processes. Aberrant vascular remodelling may result in the development of vascular pathologies such as atherosclerosis, pulmonary hypertension, aortic aneurism, as well as the intimal hyperplasia underlying saphenous vein graft failure. Accordingly, the various methods, uses or medicaments of this disclosure may be used to:
treat or prevent pulmonary hypertension;
treat or prevent aortic aneurism; and/or
treat, prevent or modulate (for example prevent or inhibit) the intimal hyperplasia underlying saphenous vein graft failure.

The various methods, uses or medicaments of this disclosure may be applied or administered to human or animal subjects, including, for example:
subjects suffering from diseases or conditions characterised by VSMC proliferation;
subjects suffering from vein graft failure/late vein graft failure;
subjects susceptible or predisposed to diseases or conditions characterised by VSMC proliferation;
subjects susceptible or predisposed to vein graft failure/late vein graft failure;
subjects undergoing or convalescing from coronary bypass surgery, including subjects undergoing or convalescing from coronary bypass surgery using saphenous veins;
subjects suffering from atherosclerosis;
subjects predisposed/susceptible to atherosclerosis;
subjects suffering from vascular injury; and/or
subjects predisposed or susceptible to from vascular injury (including vascular injury where VSMC proliferation is an important phenotype).

The term “modulate” as applied to “VSMC proliferation” may encompass any increase or decrease in the rate or occurrence/incidence of a VSMC proliferation event.

Accordingly, a miR modulator of this disclosure may be exploited as a means to either inhibit (prevent or suppress) or stimulate (encourage or increase) a VSMC proliferation event. In one teaching, a miR modulator of this disclosure may be used to inhibit (prevent or suppress) a VSMC proliferation event.

The degree of modulation affected by a miR modulator of this disclosure may be assessed relative to “normal” or “control” levels of VSMC proliferation as might occur in healthy/normal tissues not exhibiting pathology associated with aberrant VSMC proliferation.

One of skill will be familiar with the term “microRNA” (or “miR”). MicroRNAs are small non-coding RNA molecules which affect the regulation of gene expression. They are produced either from gene sequences or intron/exon sequences; many are encoded by intergenic sequences. Specific examples of suitable miR modulators are described below but a miR modulator of this disclosure may be any molecule or compound capable of either increasing or inhibiting (decreasing) the expression of a specific miR (for example one or more of the miR(s) described herein).

Within the context of this disclosure, the term microRNA or ‘miR’ embraces any one or more miRs selected from the group consisting of:
(i) miR-1827
(ii) miR-332a-3p
(iii) miR-449b-5p
(iv) miR-491-3p
(v) miR-4774-3p
(vi) miR-5681b and
(vii) miR-892b

As a such, a “miR modulator” is any compound or molecule. It has been noted that modulation of the expression (for example over-expression) of any one or more of the miRs disclosed herein (including any one or more of miRs (i)-(vii) listed above), does not (simultaneously) induce deleterious effects, such as apoptosis or senescence in VSMCs. Furthermore, the inventors have noted that modulating the expression of miR-892b results in the disclosed effects in HSVSMCs, HSVEC and PSVSMCs, simultaneously.

Accordingly, the disclosure provides modulators (for example modulators which increase the expression), of one or more of the miR(s) listed as (i)-(vii) above, for any one or more of the therapeutic applications described herein—including:
modulating VSMC proliferation;
treating or preventing diseases or conditions characterised by VSMC proliferation;
treating or preventing vein graft failure/late vein graft failure;
treating or preventing vein graft failure/late vein graft failure following surgery;
treating or preventing vein graft failure/late vein graft failure following coronary;
bypass surgery, including, for example using saphenous veins;
treating or preventing atherosclerosis;
treating or preventing vascular injury; and/or
treating or preventing vascular injury where VSMC proliferation is an important phenotype.

Of the above-listed miR(s) (aka—‘miR targets’), miR-1827, miR-332a-3p, miR-5681b and/or miR-892b have been shown not to have a significant effect on HSVEC proliferation, making these particular miRNAs of particular therapeutic interest. Without wishing to be bound by theory, targeting these miR(s) may limit unnecessary damage and/or off-target effects, to or in the host endothelium; one of skill will appreciate that this is extremely important for the various therapeutic uses described herein including, for example, the prevention of early vein graft failure.

Accordingly, the present disclosure provides modulators (for example modulators which increase expression), of one or more of:
miR-1827,
miR-332a-3p,
miR-5681b and/or
miR-892b

for use in:
modulating VSMC proliferation;
treating or preventing diseases or conditions characterised by VSMC proliferation;
treating or preventing vein graft failure/late vein graft failure;
treating or preventing vein graft failure/late vein graft failure following surgery;
treating or preventing vein graft failure/late vein graft failure following coronary;
bypass surgery, including, for example using saphenous veins;
treating or preventing atherosclerosis;
treating or preventing vascular injury; and/or
treating or preventing vascular injury where VSMC proliferation is an important phenotype.

The disclosure further provides the use of modulators for example modulators which increase expression) of one or more of miR-1827, miR-332a-3p, miR-5681b and/or miR-892b, for the manufacture of a medicament for:
modulating VSMC proliferation;
treating or preventing diseases or conditions characterised by VSMC proliferation;
treating or preventing vein graft failure/late vein graft failure;
treating or preventing vein graft failure/late vein graft failure following surgery;
treating or preventing vein graft failure/late vein graft failure following coronary;
bypass surgery, including, for example using saphenous veins;
treating or preventing atherosclerosis;
treating or preventing vascular injury; and/or
treating or preventing vascular injury where VSMC proliferation is an important phenotype.

More specifically modulators, for example modulators which increase the expression of miR-1827, miR-332a-3p, miR-5681b and/or miR-892b may be for use in methods of
modulating VSMC proliferation;
treating or preventing diseases or conditions characterised by VSMC proliferation;
treating or preventing vein graft failure/late vein graft failure;
treating or preventing vein graft failure/late vein graft failure following surgery;
treating or preventing vein graft failure/late vein graft failure following coronary;
bypass surgery, including, for example using saphenous veins;
treating or preventing atherosclerosis;
treating or preventing vascular injury; and/or
treating or preventing vascular injury where VSMC proliferation is an important phenotype;

said method comprising administering the modulators to a subject in need thereof. The modulators may be administered in a therapeutically effective amount and/or in an amount which increases the expression of the relevant miR.

The present disclosure further provides modulators (for example modulators which increase expression), of miR-892b for use in:
modulating VSMC proliferation;
treating or preventing diseases or conditions characterised by VSMC proliferation;
treating or preventing vein graft failure/late vein graft failure;
treating or preventing vein graft failure/late vein graft failure following surgery;
treating or preventing vein graft failure/late vein graft failure following coronary;
bypass surgery, including, for example using saphenous veins;
treating or preventing atherosclerosis;
treating or preventing vascular injury; and/or
treating or preventing vascular injury where VSMC proliferation is an important phenotype.

Also disclosed is the use of modulators for example modulators which increase expression) of miR-892b, for the manufacture of a medicament for:
modulating VSMC proliferation;
treating or preventing diseases or conditions characterised by VSMC proliferation;
treating or preventing vein graft failure/late vein graft failure;
treating or preventing vein graft failure/late vein graft failure following surgery;
treating or preventing vein graft failure/late vein graft failure following coronary;
bypass surgery, including, for example using saphenous veins;
treating or preventing atherosclerosis;
treating or preventing vascular injury; and/or
treating or preventing vascular injury where VSMC proliferation is an important phenotype.

Modulators, for example modulators which increase the expression of miR-892b may be for use in methods of
modulating VSMC proliferation;
treating or preventing diseases or conditions characterised by VSMC proliferation;
treating or preventing vein graft failure/late vein graft failure;
treating or preventing vein graft failure/late vein graft failure following surgery;
treating or preventing vein graft failure/late vein graft failure following coronary;
bypass surgery, including, for example using saphenous veins;
treating or preventing atherosclerosis;
treating or preventing vascular injury; and/or
treating or preventing vascular injury where VSMC proliferation is an important phenotype;

said method comprising administering the modulators to a subject in need thereof. The modulators may be administered in a therapeutically effective amount and/or in an amount which increases the expression of miR-892b.

A miR modulator for any of the uses or methods described herein may take the form of an inhibitor of one or more of the miRs described herein. The term “miR inhibitors” may comprise compounds or molecules which inhibit or reduce the expression, function and/or activity of a miR, including, for example, one or more of the miR(s) described herein.

A modulator of this disclosure may comprise a miR promoter, that is a molecule which increases the expression of the relevant miR (in a cell). The term “miR promoter” may comprise compounds or molecules which increase the expression, function and/or activity of a miR, including, for example, one or more of the miR(s) described herein.

A miR promotor may comprise, for example a miR mimic—that is a nucleic acid encoding the relevant miR for expression in a cell. One of skill will understand that when introduced into a cell, a nucleic acid encoding a specific miR, will provide an additional copy of that miR-an additional copy which supplements any native copy (or copies) expressed by the cell) such that the net result is overexpression of that miR in the cell.

A suitable miR mimic may comprise double-stranded RNA molecules mimicking mature miR duplexes.

The nucleic acid encoding the relevant miR may comprise a stem-loop miRNA.

The nucleic acid may encode any of the miRs described herein (including any of the miRs listed as (i)-(vii) herein).

The nucleic acid encoding a miR for expression may further comprise (or be operatively linked to) a promoter element and a polyA element. The promoter element and the polyA element may ‘flank’the miR encoding nucleic acid sequence.

The nucleic acid may be provided in the form of a vector for delivery to a cell.

The vector may comprise a viral vector.

The vector may comprise an adenoviral vector, e, g HAdV5 or adeno-associated viruses (e.g. AAV1, AAV2, AAV3, AAV4 or AAV5), or lentivirus.

A miR promoter, for example a nucleic acid encoding a miR, may be packaged or comprised within a viral, adenoviral or AAV5 vector.

MiR inhibitors suitable for use in this disclosure may include, for example small organic/inorganic molecules, proteins, peptides, amino acids, nucleic acids (comprising RNA, DNA and/or synthetic or peptide based nucleic acids, including PNA), carbohydrates, lipids, antibodies (including antigen binding fragments thereof) and the like.

Any of the miR modulators of this disclosure may be administered directly to a vessel wall to be treated (for example a vascular vessel wall which has been repaired through surgery and/or which shows signs of disease and/or of injury or damage). A miR modulator of this disclosure may be administered directly to a saphenous vein wall. A miR modulator of this disclosure may be administered directly to a vessel wall within, for example, the 30 minute clinical window during CABG when the SVG is available between harvesting and implantation. A miR modulator of this disclosure may be administered packaged in a vector, for example a viral (adenoviral) vector.

The disclosure provides an adenoviral vector comprising a sequence for expression of one or more of the following miR(s) in a cell:
(i) miR-1827
(ii) miR-332a-3p
(iii) miR-449b-5p
(iv) miR-491-3p
(v) miR-4774-3p
(vi) miR-5681b and
(vii) miR-892b

The disclosure provides a composition comprising a miR modulator of this disclosure and one or more excipients.

The disclosure further provides a pharmaceutical composition comprising a miR modulator of this disclosure and one or more pharmaceutically acceptable excipients.

A composition or pharmaceutical composition of this disclosure may comprise a miR modulator which is a mimic of one or more of the following miR(s):
(i) miR-1827
(ii) miR-332a-3p
(iii) miR-449b-5p
(iv) miR-491-3p
(v) miR-4774-3p
(vi) miR-5681b and
(vii) miR-892b

A composition or pharmaceutical composition may be for (i) use in or (ii) use in a method of:
modulating VSMC proliferation;
treating or preventing diseases or conditions characterised by VSMC proliferation;
treating or preventing vein graft failure/late vein graft failure;
treating or preventing vein graft failure/late vein graft failure following surgery;
treating or preventing vein graft failure/late vein graft failure following coronary;
bypass surgery, including, for example using saphenous veins;
treating or preventing atherosclerosis; and/or
treating or preventing vascular injury.

Were the composition or pharmaceutical composition is for use in a method of treatment, the composition or pharmaceutical composition may be administered to a subject (as defined herein) in need thereof.

Additionally, a composition or pharmaceutical composition may be used in the manufacture of a medicament for
modulating VSMC proliferation;
treating or preventing diseases or conditions characterised by VSMC proliferation;
treating or preventing vein graft failure/late vein graft failure;
treating or preventing vein graft failure/late vein graft failure following surgery;
treating or preventing vein graft failure/late vein graft failure following coronary;
bypass surgery, including, for example using saphenous veins;
treating or preventing atherosclerosis; and/or
treating or preventing vascular injury.

### DETAILED DESCRIPTION

The present disclosure will now be described ion further detail with reference to the following figures which show:

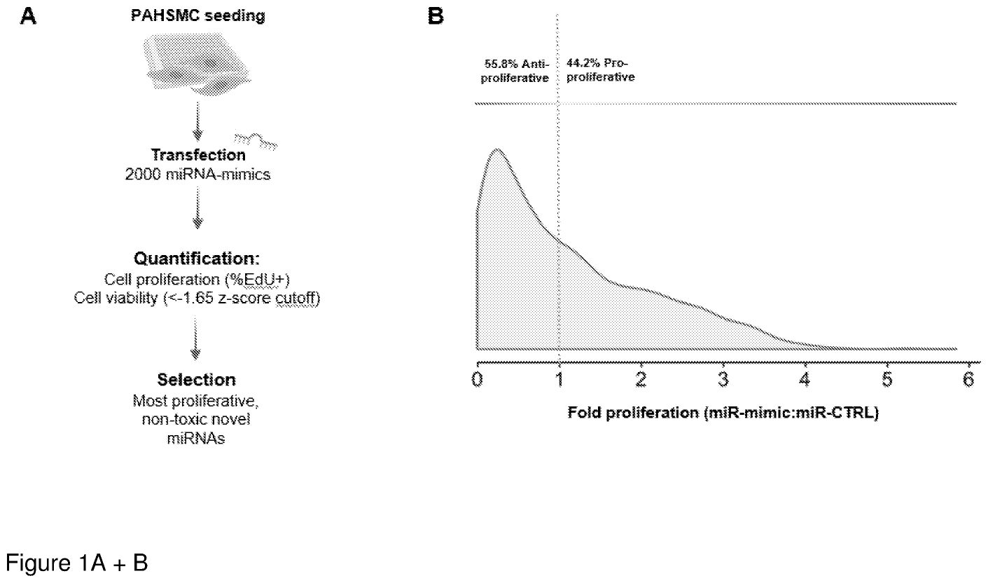

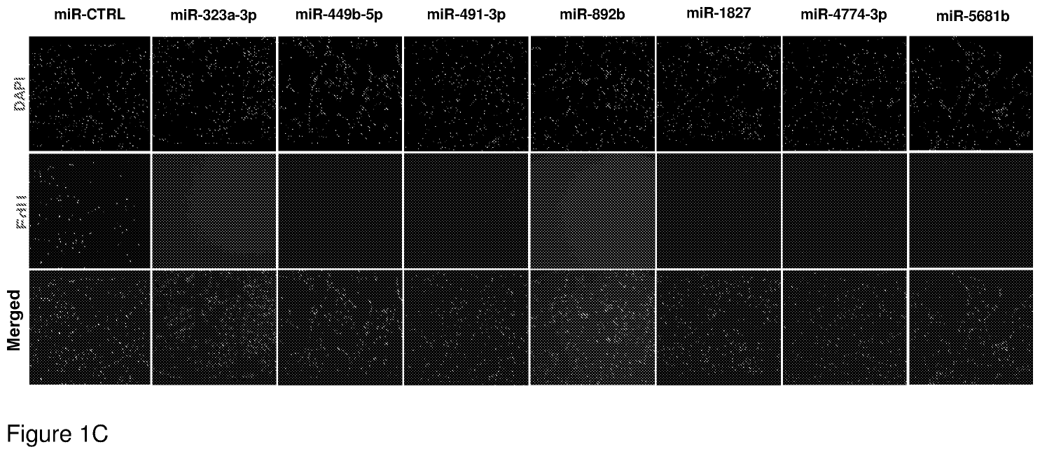

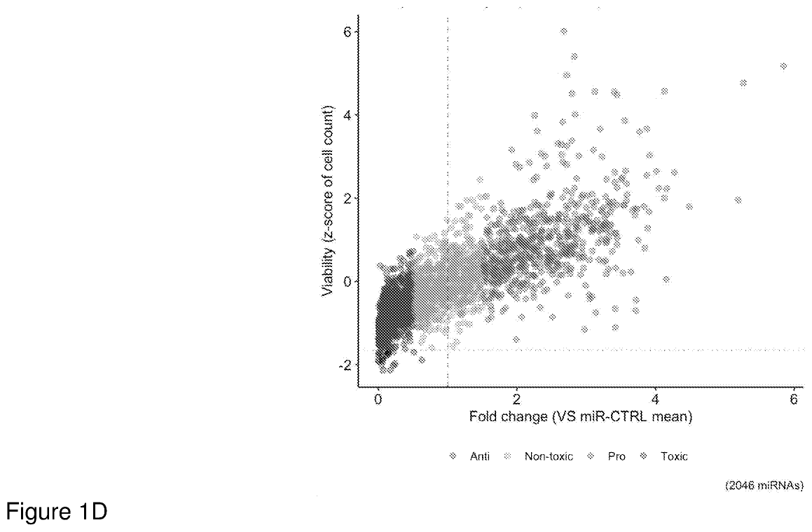

FIG. 1: High-throughput miRNA screen identifies novel miRNAs regulating VSMC proliferation. A, Schematic of high-throughput miRNA screen design. B, Density plot showing the distribution of miRNAs tested in the screen based on their effect on VSMC proliferation expressed as fold change following normalisation to miR-CTRL-transfected VSMCs. C, Microscopy images of human VSMCs stained with DAPI (blue) and EdU (green) following treatment with each of the 7 candidate miRNA, as well as miR-CTRL. D, Scatter plot of fold change in PASMC proliferation (miR-mimics vs miR-CTRL). Each point represents one miRNA. Anti=Anti-proliferative, Pro=Pro-proliferative.

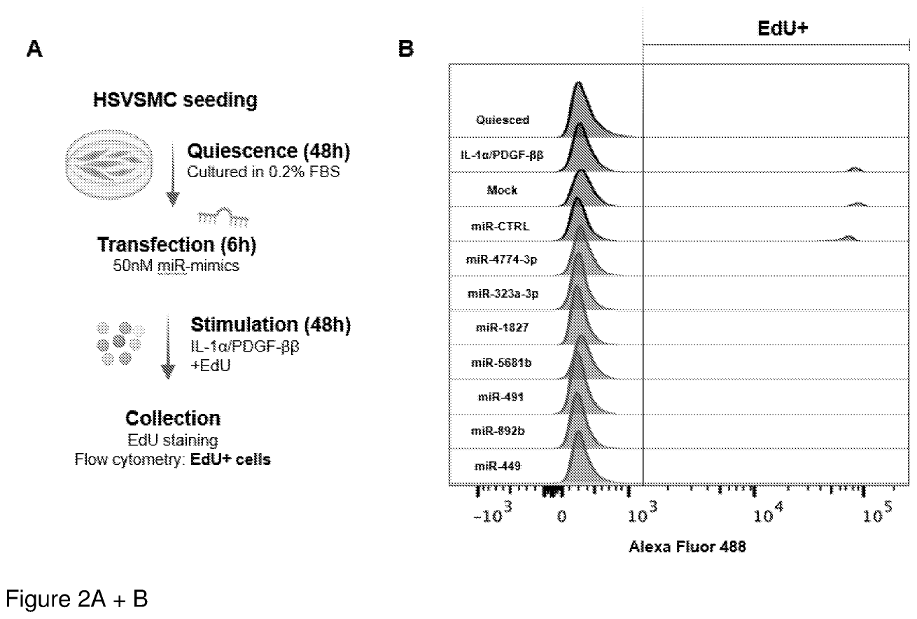

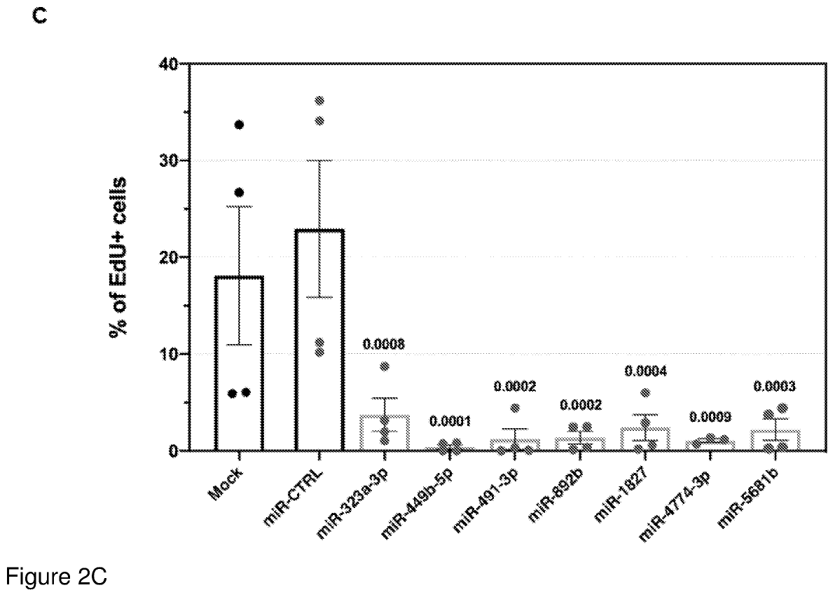

FIG. 2: In vitro assessment of the effect of 7 novel candidate miRNA overexpression in HSVSMC proliferation. A, Schematic of experimental design for miRNA overexpression using miR-mimics in HSVSMCs. B, Representative fluorescence-activated cell sorting (FACS) plots showing the 5-ethynyl-2′-deoxyuridine (EdU) uptake in HSVSMCs transfected with the 7 miRNA mimics or miR-CTRL. The gate (vertical line) indicates EdU-positive cells. C, Flow cytometric quantification of EdU incorporation in HSVSMCs following transfection with each of the novel 7 candidate miRNA and subsequent stimulation with Platelet-derived Growth Factor ββ (PDGF-ββ)/Interleukin 1-α (IL-1α) (n=3-4). Statistical analysis and p-values were obtained using Mixed-Effects analysis and Dunnett's test for multiple corrections, where the means of all columns were always compared to the mean of the miR-CTRL column. N numbers correspond to distinct biological replicates. P-values are included on the graph.

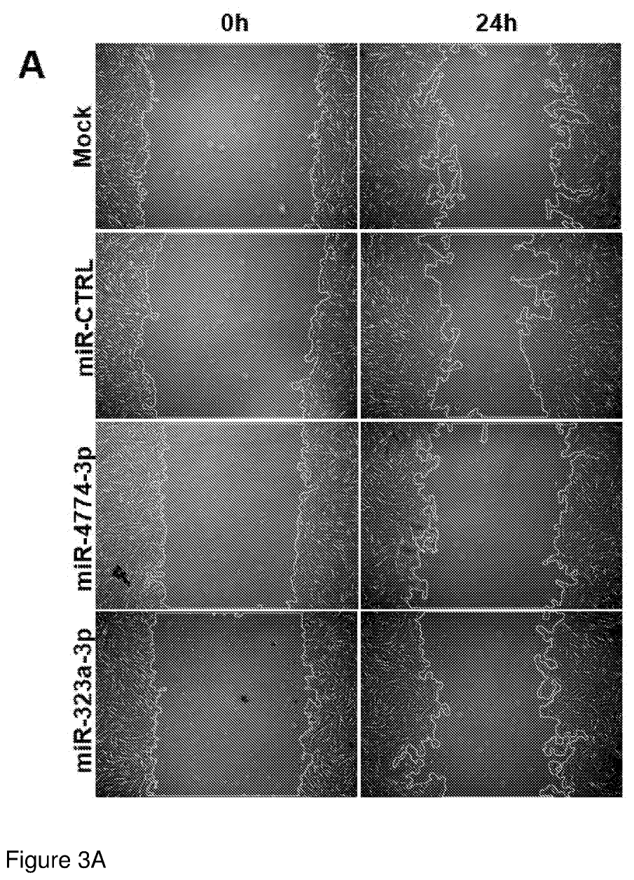

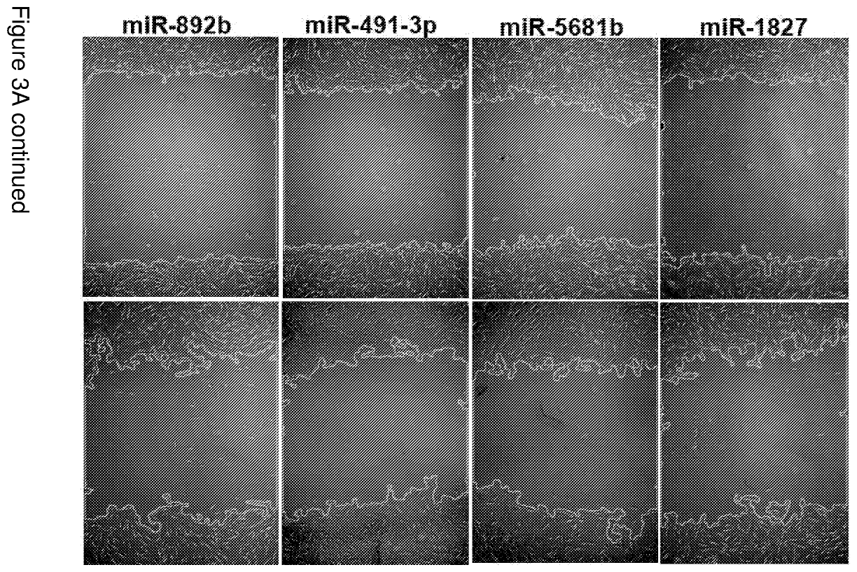

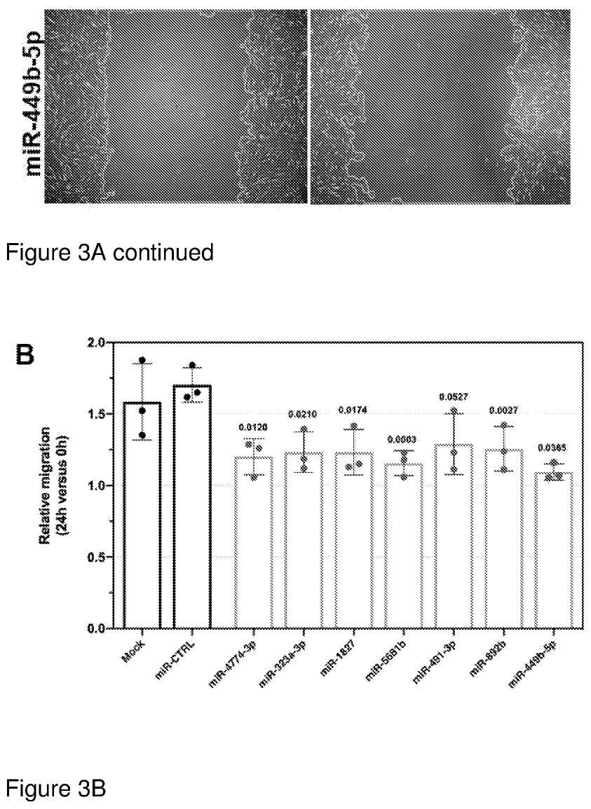

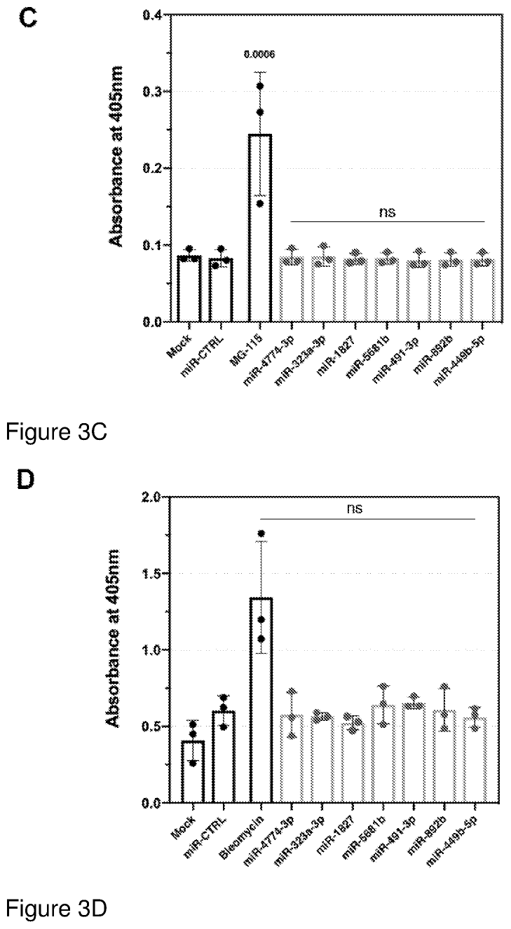

FIG. 3: In vitro assessment of the effect of 7 novel candidate miRNA overexpression in HSVSMCs. A, Representative images from scratch assay showing IL-1a/PDGF-bb-stimulated HSVSMCs at 0-and 24-hours from scratch following transfection with miRNA mimics, miR-CTRL, and Mock control (n=3). B, Relative migration distance quantification of IL-1a/PDGF-bb-stimulated HSVSMCs obtained via the ImageJ MRI wound healing tool (n=3). C, Quantification of Caspase-3 activity following miRNA overexpression (measured at 405 nm, n=3). The proteasome inhibitor MG-115 was used as a positive control for apoptosis induction. D, Quantification of Senescence Associated (SA) β-galactosidase (β-gal) activity following miR-mimic transfection (measured at 405 nm, n=3). Bleomycin (1 μg/mL) was used as a positive control for senescence induction. Statistical analysis and p-values in all cases were obtained using the Iman-Conover rank for transformation of non-normal data, followed by repeated-measures (RM)-ANOVA and Dunnett's test for multiple corrections, where the means of all columns were always compared to the mean of the miR-CTRL column. N numbers correspond to distinct biological replicates. P-values are included on the graph. “Ns” stands for “non-significant”

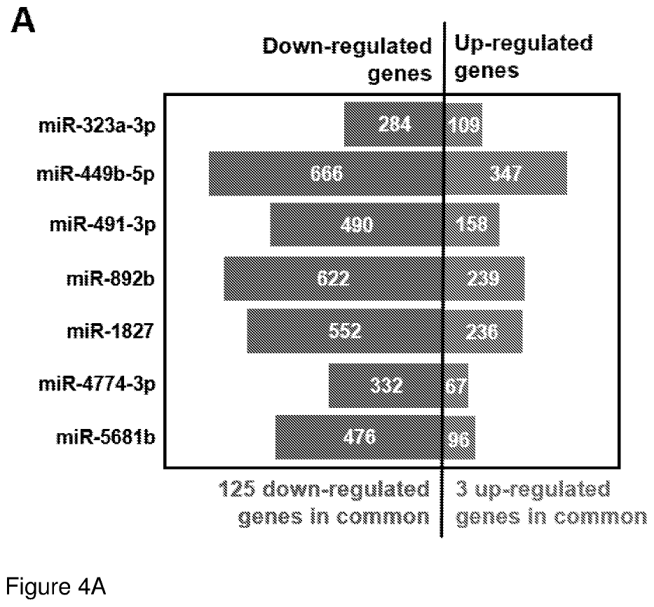

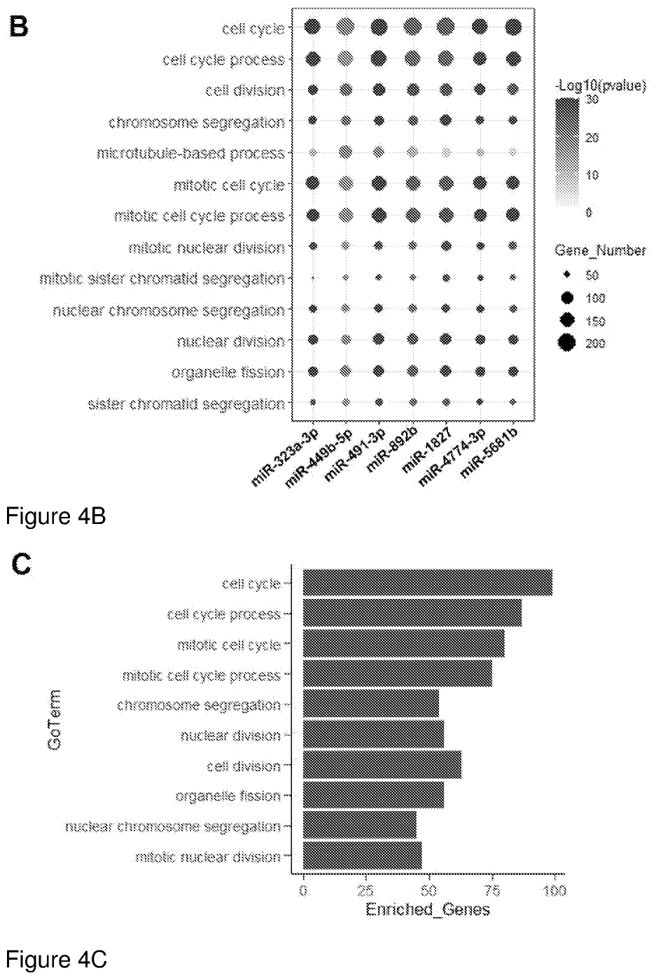

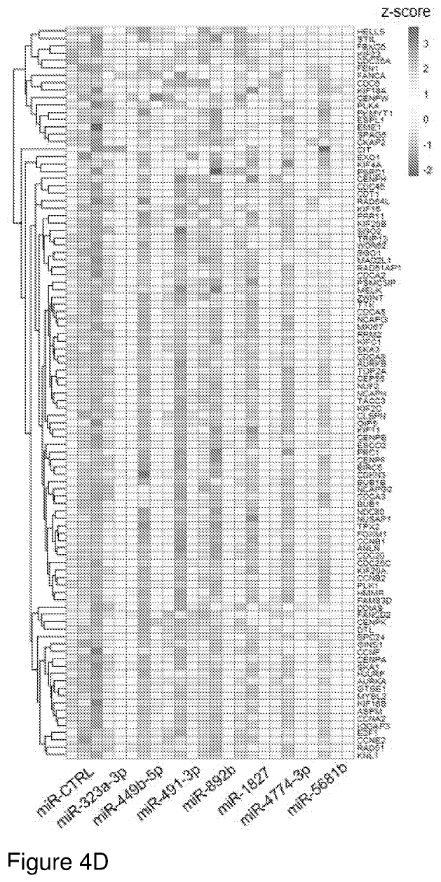

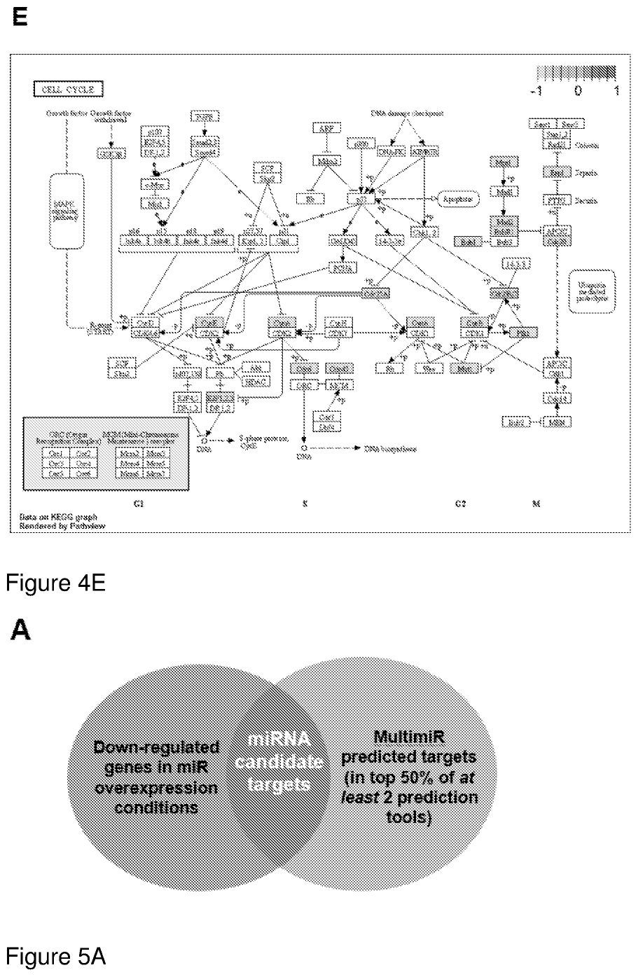

FIG. 4: RNA-sequencing and bioinformatics analysis revealed candidate targets of the 7 candidate miRNA associated with cell cycle, migration and glycosylation. A. Number of up-regulated and down-regulated differentially expressed genes following overexpression of each miRNA. B, Gene Ontology (GO) terms analysis showing GO terms for all differentially expressed genes due to overexpression of each miRNA, ranked based on enrichment. C, Most significant Gene Ontology (GO) terms of the 125 commonly down-regulated genes in 4A, ranked by fold enrichment. D. Heatmap (as Z score of log2[FPKM+1]) of all 99 genes out of the 125 commonly downregulated genes in 4A that are included in the GO term annotated as “Cell Cycle”. E, Schematic of the KEGG cell cycle pathway, with genes significantly downregulated by candidate miRNA overexpression highlighted in green.

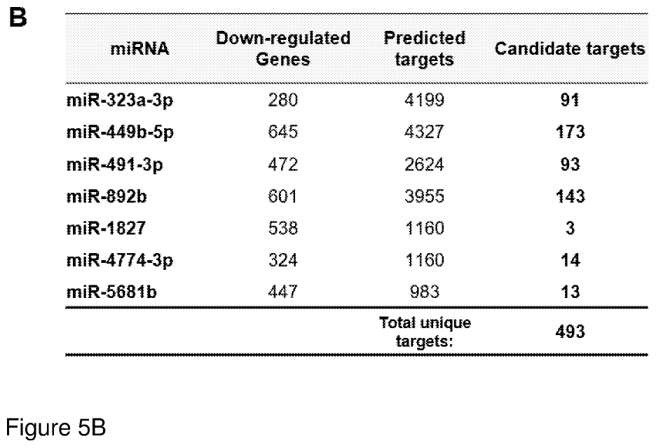

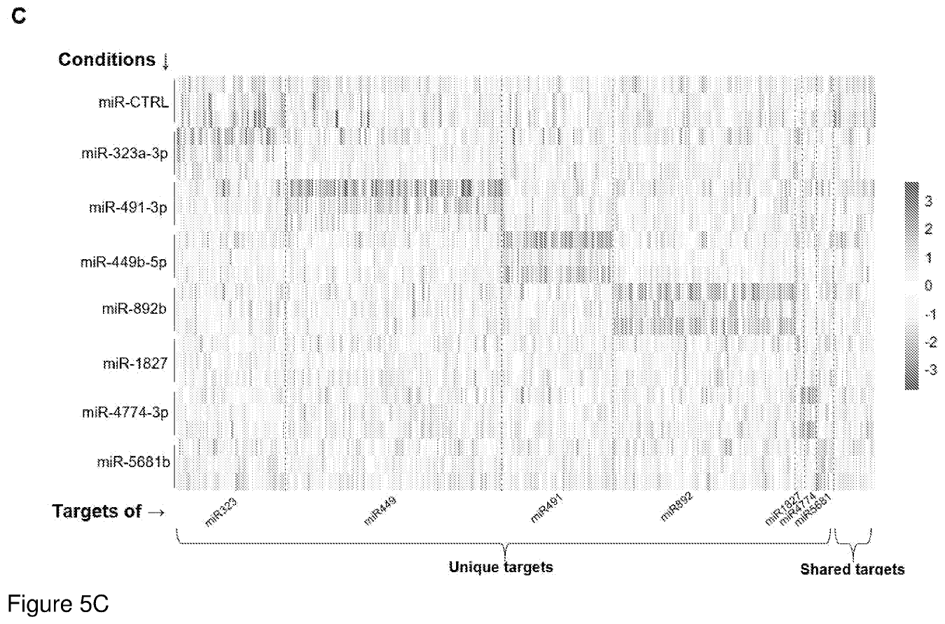

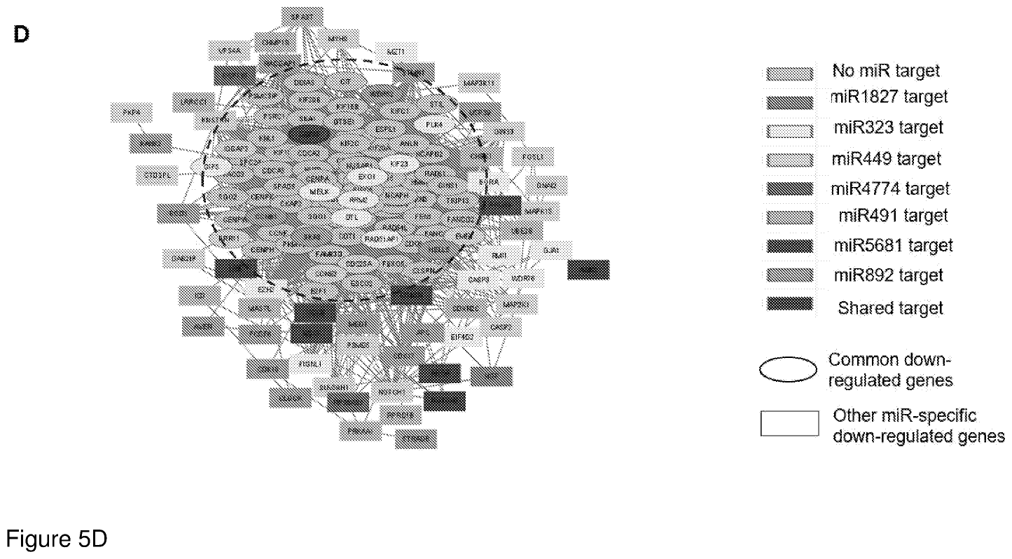

FIG. 5: The miRNAs function through distinct sets of candidate targets which include cell-cycle associated genes. A, Schematic of bioinformatics pipeline used to identify likely functional miRNA-mRNA interactions. B. Table showing the number of: i. down-regulated genes, ii. compiled miRNA predicted targets from the multimiR tool, and iii. The intersection of i. and ii., which represents candidate targets. C. Heatmap (as Z score of log2[FPKM+1]) of all candidate miRNA targets listed in 5B, ordered horizontally based on the miRNA they are putatively targeted by, based on pipeline described in 5A. D, STRING schematic of the association between the common down-regulated genes and the candidate targets of each miRNA that are annotated under the “Cell Cycle” GO term (168 genes).

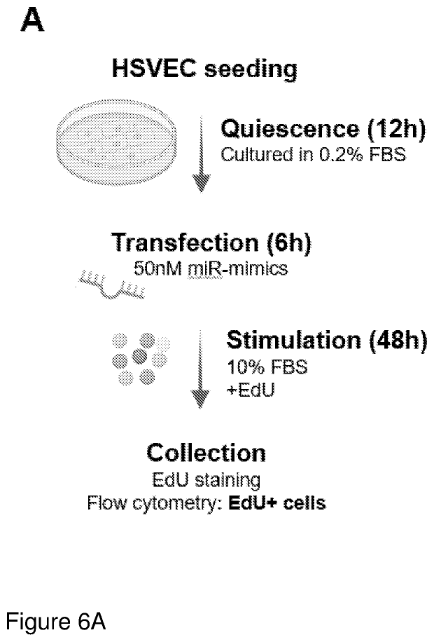

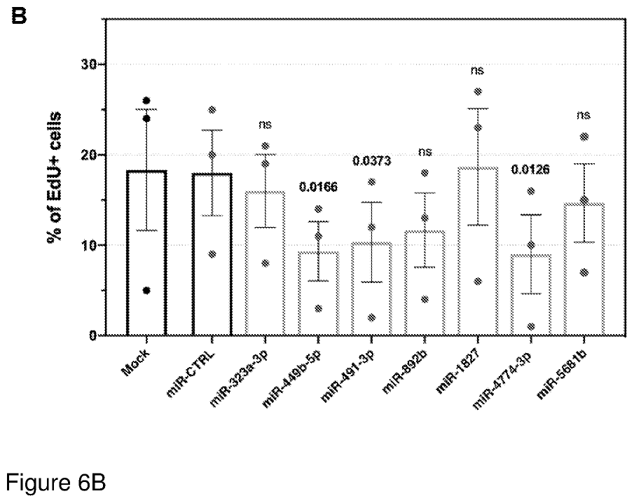

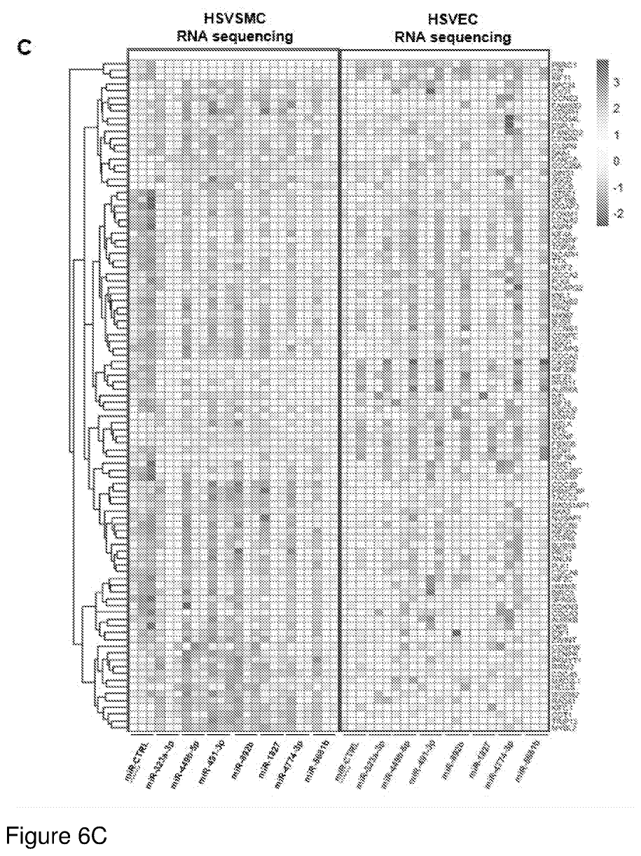

FIG. 6: In vitro assessment of the effect of the novel 7 candidate miRNA in HSVECs. A, Schematic of experimental design for miRNA overexpression using miR-mimics in HSVECs. B, Flow cytometric quantification of EdU incorporation in HSVECs following transfection with each of the novel 7 candidate miRNA and subsequent stimulation with 10% FBS (n=3). Statistical analysis and p-values were obtained using the Iman-Conover rank for transformation of non-normal data, followed by RM-ANOVA and Dunnett's test for multiple corrections, where the means of all columns were always compared to the mean of the miR-CTRL column. N numbers correspond to distinct biological replicates. P-values are included on the graph. “Ns” stands for “non-significant”. C, Heatmap (as Z score of log2[FPKM+1]) of the cell cycle process-enriched set of 125 commonly downregulated genes from FIG. 4A.

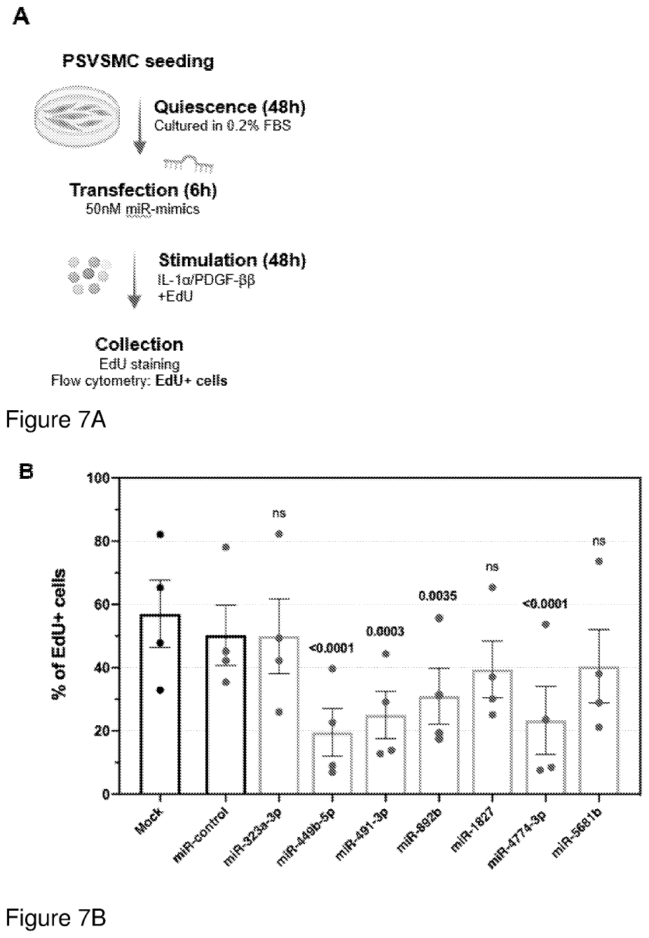

FIG. 7: In vitro assessment of the effect of the novel 7 candidate miRNA in PSVSMCs. A, Schematic of experimental design for miRNA overexpression using miR-mimics in PSVSMCs. B, Flow cytometric quantification of EdU incorporation in PSVSMCs following transfection with each of the novel 7 candidate miRNA and stimulation with 10% FBS (n=4). Statistical analysis and p-values were obtained using the Iman-Conover rank for transformation of non-normal data, followed by RM-ANOVA and Dunnett's test for multiple corrections, where the means of all columns were always compared to the mean of the miR-CTRL column. N numbers correspond to distinct biological replicates. P-values are included on the graph. “Ns” stands for “non-significant”

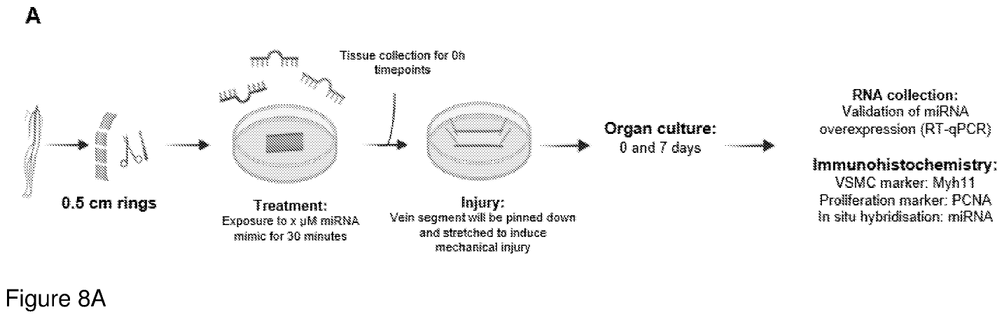

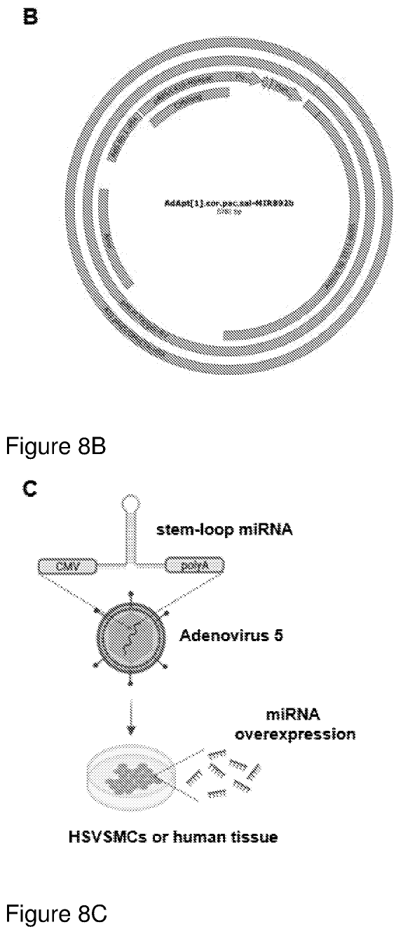

FIG. 8: In vitro and ex vivo Adenovirus-mediated overexpression of miRNA candidates. A, Schematic representation of the miRNA overexpression strategy for ex vivo saphenous vein organ culture. B, Vector map of adenovirus serotype 5 (Ad5)expressing the stem-loop of the candidate miRNA (human miR-892b in the schematic was taken as example) under the Cytomegalovirus promoter (CMV), including a poly adenosine (polyA) tail at the end of the miRNA stem-loop sequence. C, Schematic representation of the infection strategy in primary human saphenous vein smooth muscle cells (HSVSMCs) or human vein tissue. The virus expressing the microRNA of interest, will be incubated with cells or tissue at a specific multiplicity of infection (MOI) to induce the overexpression of mature microRNA following processing of the stem-loop. Moreover, an adenoviral vector expressing LacZ will be used as negative control.

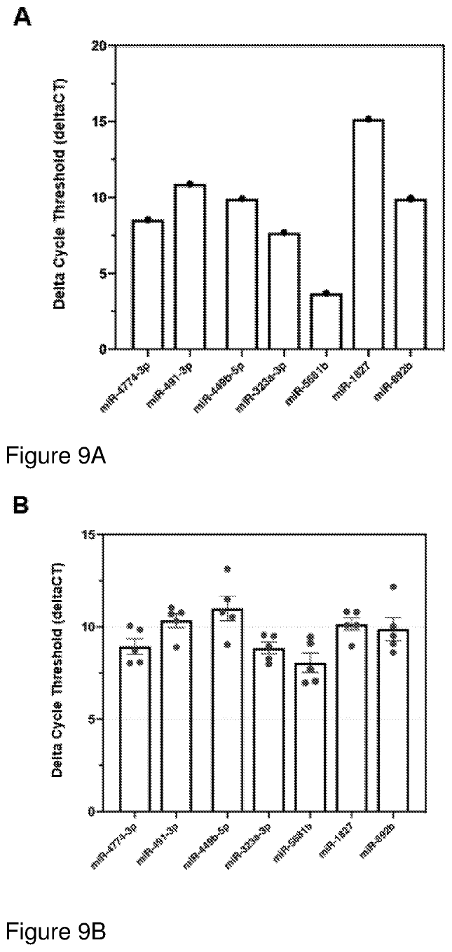

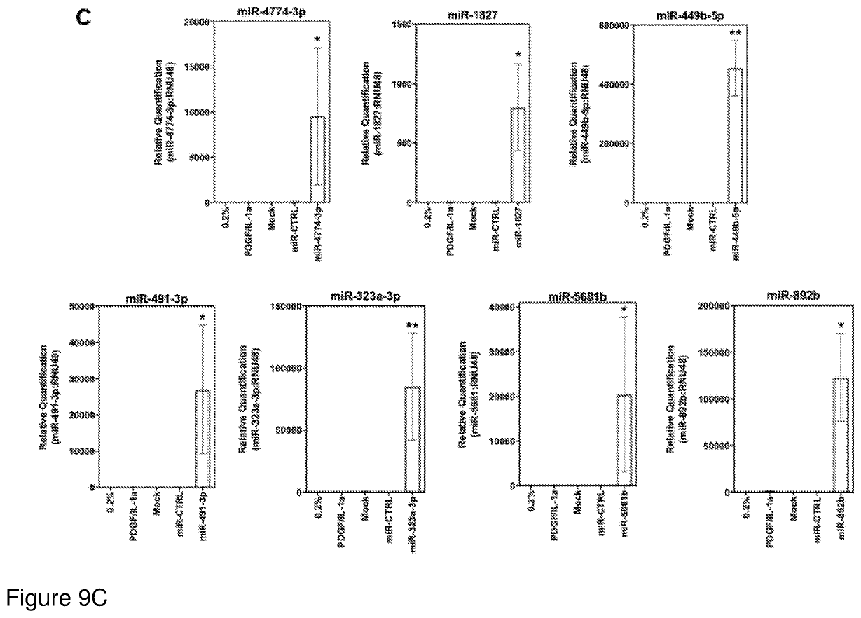

FIG. 9: Expression of candidate miRNA in basal HSVSMC, saphenous vein tissue and following mimic overexpression in HSVSMCs. A, Basal expression of each of the selected miRNAs in quiesced primary HSVSMC quantified by reverse-transcription quantitative PCR (RT-qPCR) in HSVSMCs and visualised as deltaCt after normalisation using engogenous expression control gene RNU48. B, Basal expression of each of the selected miRNAs in RNA from human saphenous vein tissue lysates and visualised as deltaCt after normalisation using engogenous expression control gene RNU48. C, Relative quantification of each of the selected miRNA candidates in primary HSVSMCs following transfection with 50 nM of miRNA mimics. 0.2%=48 h in basal conditions following quescence, PDGF/IL1a=stimulation with IL-1α/DGF-ββ for 48 h, Mock=mock transfected HSVSMC.

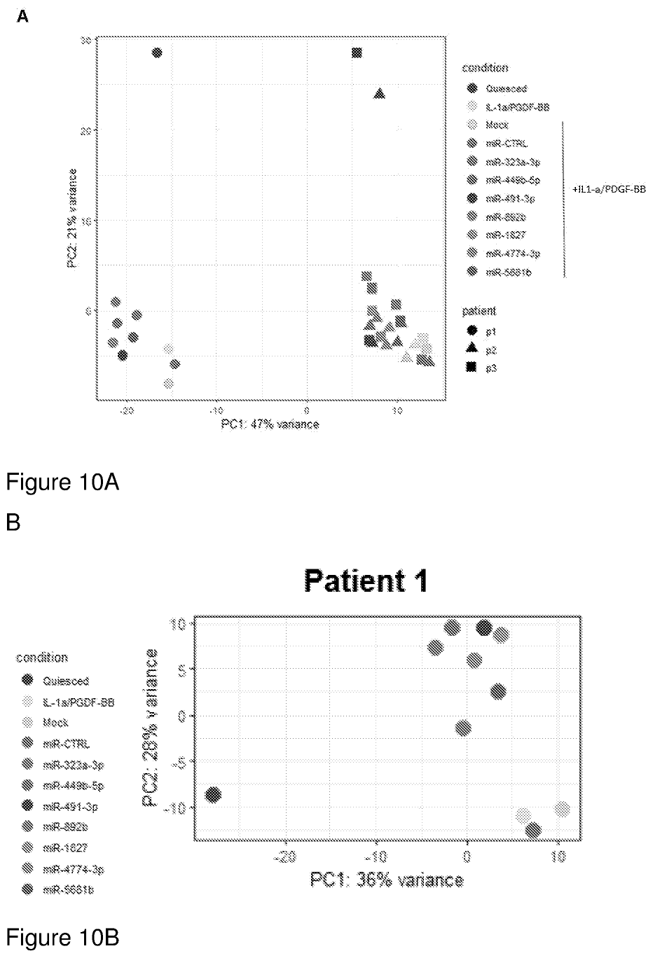

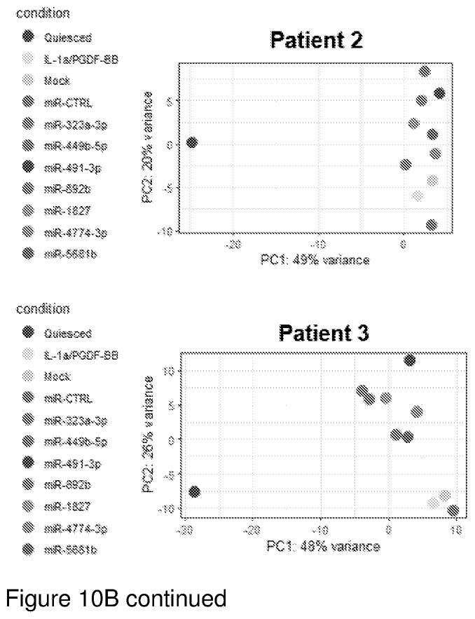

FIG. 10: Principal component analysis of the RNA-sequencing dataset in HSVSMCs. A, Graph showing the principal component analysis (PCA) of the RNA-sequencing dataset in HSVSMCs with miRNA overexpression, as well as of controls, done for all patients. Different colours represent paired samples: green hues correspond to controls, red hues correspond to miRNA-overexpression conditions, whereas the quiesced HSVSMC condition is represented in purple. Different shapes denote different patient origins of the HSVSMCs used. B, PCA plots for each patient individually. Samples are colour coded similarly to FIG. 10A.

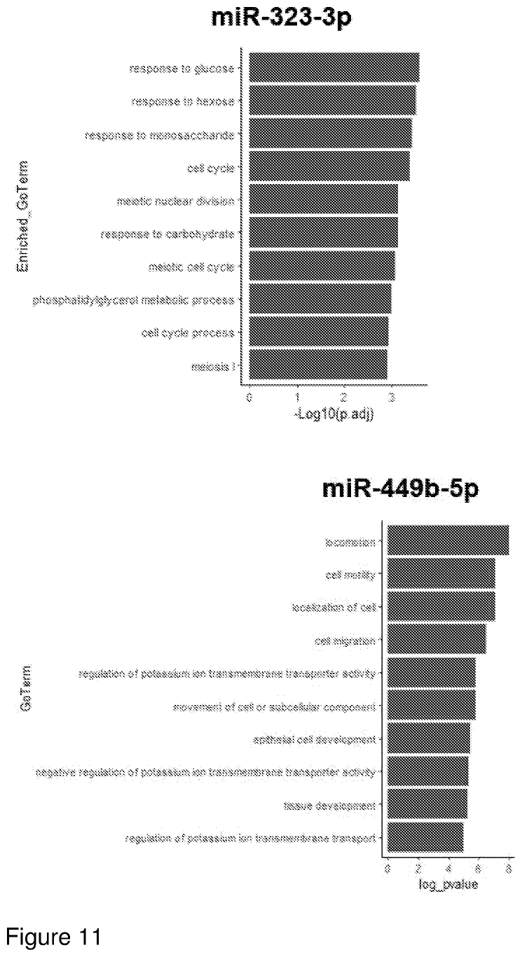

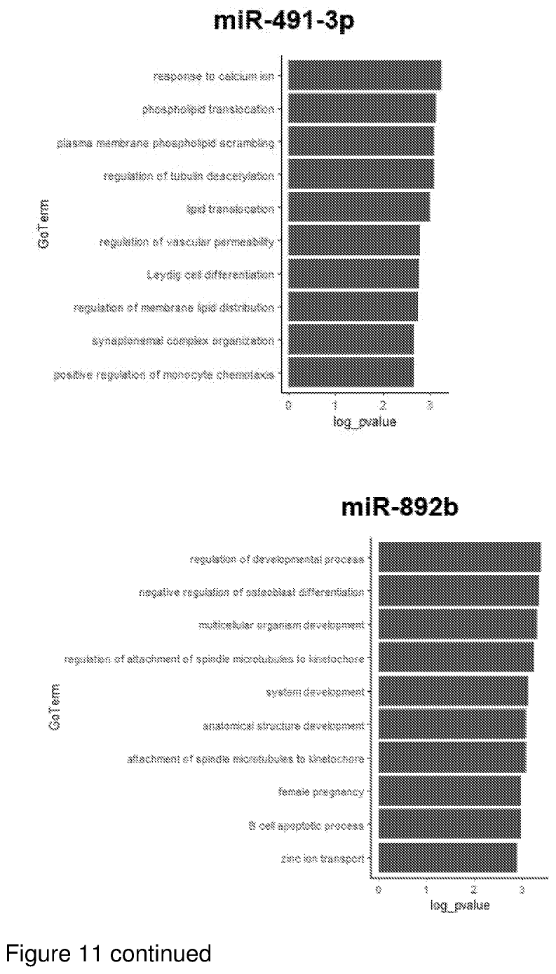

FIG. 11: Top GO terms of miR-323a-3p, miR-449b-5p, miR-491-3p and miR-892b candidate targets (>20 genes) Graphs showing the 10 most significantly enriched GO terms for targets of miR-323a-3p, miR-449b-5p, miR-491-3p and miR-892b.

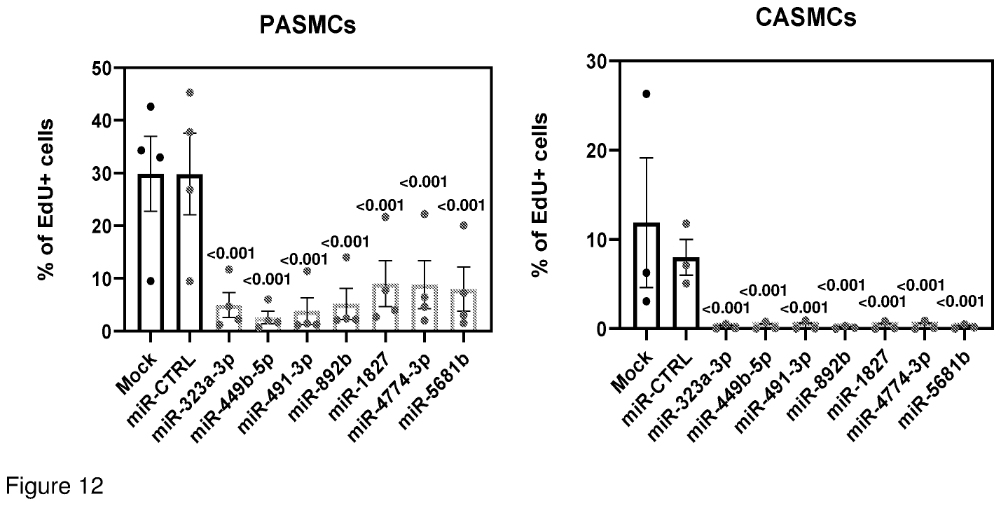

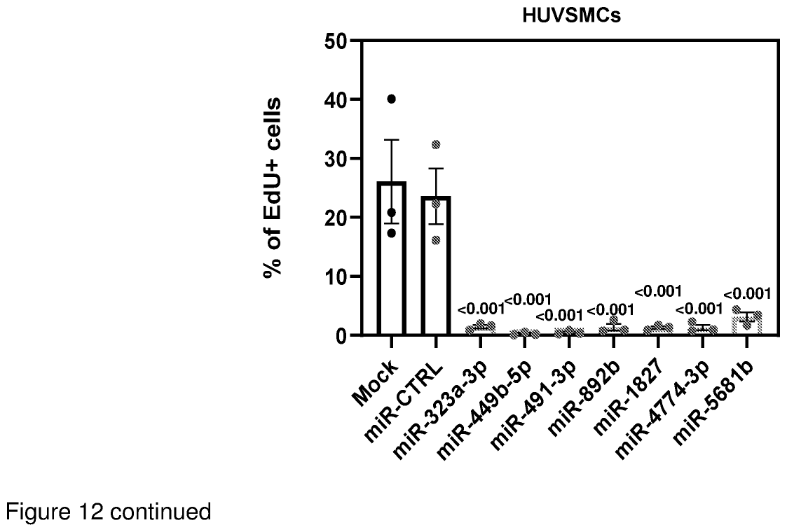

FIG. 12: Validation of the screen in PASMCs, CASMCs and HUVSMCs. High-throughput microscopy imaging quantification of EdU incorporation in PASMCs (n=4), CASMCs (n=3) and HUVSMCs (n=3) transfected with the 7 miRNA mimics or miR-CTRL, as well as the “mock” transfection control. Statistical analyses were done using Repeat Measures (RM) ANOVA and Dunnett's test for multiple corrections. P-values for the comparison between miRNA mimic treatment and miR-CTRL treatment are included on the graph. N numbers correspond to distinct biological replicates.

### EXAMPLES

### Functional Screening Identifies Novel miRNAs Selectively Inhibiting Vascular Smooth Muscle Cell Proliferation

### Abstract

Aberrant vascular smooth muscle cell (VSMC) proliferation in response to vascular injury is a key driver of pathological remodelling in the vessel wall, a major underlying factor of vascular disease. Therefore, targeting VSMC proliferation represents a promising therapeutical opportunity.

### Objectives

To systematically identify novel miRNA that selectively exert an antiproliferative effect in VSMCs and to study their therapeutic value in pathological vascular remodelling.

### Methods and Results

A library of 2000 human miRNA-mimics was assessed for their effect on modulating VSMC proliferation in a high-throughput in vitro functional screen. The following were selected for further assessment: miR-1827, miR-4774-3p, miR-5681b, miR-449b-5p, miR-491-3p, miR-323a-3p and miR-892b. Functional validation of those 7 candidates in primary human saphenous vein smooth muscle cells (HSVSMCs) showed that their overexpression was able to significantly reduce proliferation without inducing apoptosis nor senescence, with 6 of them also significantly reducing migration. RNA sequencing of HSVSMCs following transfection with the respective miRNA mimics, revealed that overexpression of the 7 candidate miRNAs resulted in transcriptomic changes significantly associated with cell cycle regulation. Through interrogating candidate targets for each miRNA, we observed that, individually, the miRNAs function via distinct mechanisms from one another which converge at the regulation of the cell cycle process.

### Introduction

Vascular remodelling is an essential process of adaptive structural change in the vessel wall. It involves changes in vascular wall thickness, which confers elevated vascular resistance in response to pathological, haemodynamic, or iatrogenic injurious cues. However, this process is often aberrant, resulting in the development of vascular pathologies such as atherosclerosis, pulmonary hypertension, aortic aneurism, as well as the intimal hyperplasia underlying saphenous vein graft failure. Integral to the aetiology of the vascular remodelling process is the switch in resident VSMCs from a differentiated and quiescent to a de-differentiated, pro-proliferative and pro-migratory phenotype¹⁻³. On the molecular level, this is induced by the release of growth factors and inflammatory cytokines following injury of the endothelial cell layer in the vessel lumen and the subsequent inflammatory response, such as platelet-derived growth factor BB (PDGF-BB) and interleukin-1a (IL-1A)⁴. This renders targeting of VSMC proliferation an attractive therapeutic strategy for preventing adverse vascular remodelling in response to injury. Therapeutic approaches based on reducing VSMC proliferation have been successful pre-clinically and shown promising clinical results, as demonstrated by animal studies and clinical trials testing the use of anti-proliferative pharmacological agents in drug-eluting stents used for coronary angioplasty⁵. The pitfall of these approaches, however, has been interference with re-endothelisation, a process integral in countering the subsequent pathological vascular remodelling events that initiate following endothelial cell injury and denudation. In saphenous vein grafts, a decoy of E2F family transcription factors, which are activators or cell-cycle associated genes, successfully prevented intimal hyperplasia preclinically in vivo. Despite this, its associated clinical trial failed due to no observed reduction in vein graft failure events⁶. These results highlight the need for the development of novel therapeutic approaches for vascular remodelling-associated vascular disease.

MicroRNAs (miRNAs) are small non-coding RNA molecules (20-24 nucleotides in length) that regulate gene expression through imperfect base-pairing with regions in the 3′UTR of target messenger RNA (mRNA), inducing their translational repression or degradation. MiRNAs have been shown to play critical roles in a range of biological contexts⁷. In cardiovascular physiology, dysregulation of several miRNAs has been implicated in the development of disease associated VSMC phenotypic switch by regulating various homeostatic processes of vascular cells⁸, with single miRNAs often regulating VSMC function across multiple vascular disease contexts. A notable example of such regulation can be given by the SMC-enriched miR-143/145 cluster, which has a well-described role in neointimal lesion formation, pulmonary arterial hypertension, and atherosclerosis^{9,10}. Individual miRNAs can regulate the expression of multiple target mRNA transcripts that are often implicated in the same signalling pathway or biological process. The ability to modulate their abundance using miRNA mimics, inhibitors, or viral vector-mediated overexpression of miRNA loci to induce downstream transcriptomic changes has given rise to the development of novel therapeutic approaches in multiple disease contexts, including cardiovascular disease^{11,12}. In the past, there have been numerous attempts at modulating the endogenous levels of previously-studied VSMC miRNA-regulators with the aim to achieve attenuation of the effect of vascular injury¹³. However, none of them have progressed into clinical translation.

### Results

Functional Screening Identifies Mirnas that Effectively Block Early-Passage VSMC Proliferation.

In vitro high-throughput miRNA screening was performed using a library containing mimics of 2000 different human miRNAs annotated in miRbase v21 to identify miRNAs exerting antiproliferative effects in VSMCs (FIG. 1A). Early-passage primary human VSMCs were transfected with the library of miRNA mimics for 72 hours in basal condition. Next, high-content fluorescent image analysis was performed to measure proliferation by quantifying the incorporation of the thymidine analogue 5-ethylnyl-2′-deoxyuridine (EdU), as well as cell viability by assessing cell number. Out of all miRNAs tested, 1141 (55.8%) reduced proliferation, whereas 903 (44.2%) of miRNAs promoted it, with maximum fold increase reaching 5.84 versus transfection with miRNA mimic control (miR-CTRL) (FIG. 1B). Fourteen miRNAs reduced proliferation to 0%, out of which seven were taken forward as the most interesting candidates based on:
i) their ability to not significantly reduce cell viability, as identified based on a deviation of over −1.65 standard deviations from the mean (z-score of cell number>−1.65, p=0.1), as well as
ii) their novelty in the literature in any cardiovascular context.

These miRNAs are: miR-1827, miR-4774-3p, miR-5681b, miR-449b-5p, miR-491-3p, miR-323a-3p and miR-892b (FIG. 1C).

### Overexpression of Chosen Mirna Candidates Reduces Proliferation and Migration of Stimulated Hsvsmcs, Without Inducing Apoptosis nor Senescence.

The saphenous vein graft is suited for ex vivo therapeutic interventions during coronary artery bypass surgery. This is due to a therapeutic window created between harvesting and implantation that might allow ex vivo manipulation to prevent remodelling, as realised in gene therapy studies from our laboratory¹⁴. Therefore, this clinical setting was utilised for the evaluation of the effect of the 7 miRNA candidates identified through the initial screen. The endogenous expression profile of the 7-candidate miRNAs was characterised in primary human saphenous vein VSMCs (HSVSMCs) in basal conditions, as well as in whole human saphenous vein tissue, using reverse-transcription quantitative polymerase chain reaction (RT-qPCR). As seen in FIGS. 9A-B, no, or very low expression of each of the 7-novel miRNA was observed in HSVSMCs and human saphenous vein, respectively, as demonstrated by the delta cycle threshold (Ct) value following normalisation with housekeeping gene RNU48 (higher deltaCt values indicate lower expression). Furthermore, RT-qPCR in HSVSMCs following transfection with mimics confirmed significant overexpression of the miRNAs compared to miR-CTRL (FIG. 9B).

Next, it was tested whether candidate miRNA overexpression in stimulated HSVSMCs results in a reduction of proliferation. Following a 48-hour serum-starvation to induce quiescence, HSVSMCs were transiently transfected with mimics of the seven miRNAs, before a 48 h stimulation with IL-1α/PDGF-ββ (FIG. 2A). Flow cytometric quantification of EdU incorporation (FIG. 2B) showed significant decreases in IL-1α/DGF-ββ-induced proliferation ranging from 83.7 to 98.2% after transfection with the seven miRNA mimics, compared to miR-CTRL (FIG. 2C). This reduction in proliferation in HSVSMCs is consistent with and further corroborating with the anti-proliferative effects that the candidate miRNAs exerted on human SMCs in the high throughput screen.

We additionally sought to evaluate whether overexpression of the novel candidate miRNAs affects migration in HSVSMCs. As seen in FIG. 3A-B, six miRNAs significantly reduced the migration rate of IL-1a/PDGF-bb-stimulated HSVSMC, assessed by scratch wound assay.

To test whether the candidate miRNAs can induce deleterious phenotypic effects, which could account for the observed reductions in proliferation and migration in HSVSMC, the effect of miRNA overexpression on apoptosis and senescence was measured. None of the miRNAs were found to induce apoptosis compared to treatment with the proteasome inhibitor MG-115¹⁵, as measured by Caspase-3 activity in HSVSMC (FIG. 3C). This is in agreement with the non-significant effect in cell number observed in the miRNA screen, which would suggest a putative toxic effect induced by the miRNAs. Furthermore, senescence-associated (SA) β-galactosidase activity did not show an increasing trend compared to bleomycin-treated¹⁶ HSVSMCs (FIG. 3D). These results demonstrate that overexpression of the candidate miRNAs reduces IL-1α/PDGF-ββ-induced HSVSMCs proliferation and migration, without causing HSVSMC apoptosis nor senescence.

RNA-sequencing in HSVSMCs reveals the regulation of a common network of cell cycle genes following overexpression of all 7 miRNAs.

To understand the effect of the 7 miRNA candidates in HSVSMCs at the transcriptomic level, RNA-sequencing was performed on IL-1α/PDGF-ββ-stimulated, proliferating HSVSMCs4 with overexpression of the candidate miRNA versus miR-CTRL, as well as on quiescent HSVSMC samples. Principal component analysis (PCA) showed distinct clustering of IL-1A/PDGF-BB-stimulated HSVSMCs versus quiesced HSVSMCs (FIG. 10). The PCA analysis also showed no overlap between miRNA mimic-treated HSVSMCs and their relative IL-1A/PDGF-BB controls (represented using green hues in FIG. 10), with miRNA mimic-overexpression HSVSMC samples (in red hues) remaining distinct from quiescent HSVSMCs. This analysis reveals how overexpression of candidate miRNA results in distinct transcriptomic changes of HSVSMCs described hereinafter. FIG. 10B highlights a similar pattern observed between the patients, with the effect of IL-1A/PDGF-BB stimulation (both with and without miRNA overexpression) in HSVSMCs clearly separating those samples from quiesced HSVSMCs, with an additional separation between samples with simultaneous IL-1A/PDGF-BB stimulation and miRNA overexpression, versus IL-1A/PDGF-BB-stimulated controls.

Differential expression analysis was performed to identify genes that were significantly differentially expressed at least two-fold between miRNA mimic overexpression and miR-CTRL samples, as previously described¹⁷. Due to sample separation based on patient origin in the PCA plot (FIG. 10), we corrected for patient variance in the differential gene expression analysis and detected between 389 and 1033 differentially expressed genes in total following miRNA overexpression (FIG. 4A). For each list of differentially expressed genes corresponding to the overexpression of each candidate miRNA, Gene Ontology analysis was performed and discovered that the top 10 enriched Gene Ontology (GO) terms for each miRNA overexpression were exclusively related to cell cycle-related processes (FIG. 4B)^{18,19,20}. This is in agreement with the strong effect of the candidate miRNA overexpression on HSVSMC proliferation and reveals that the 7 miRNAs appear to regulate similar anti-proliferative pathways downstream.

Due to the regulation of similar pathways by the 7 miRNAs suggesting regulation of common genes, we assessed the overlap of the differentially regulated genes for each miRNA. We identified 3 commonly up-regulated genes and 125 commonly down-regulated genes by all 7 candidate miRNAs. Canonically, miRNAs downregulate mRNA expression 7, therefore we focused on the 125 commonly down-regulated genes for investigating the transcriptomic changes shared by the 7-candidate miRNAs. GO term enrichment analysis found the most enriched GO terms of these 125 common genes to be related to cell cycle and mitosis (FIG. 4C). We found 99 out of the 125 down-regulated genes to be associated with the “Cell Cycle” GO Term annotation (FIG. 4D), while 17 of them were associated with the KEGG “cell cycle pathway”, which includes the main components required for cell cycle progression (FIG. 4E)^{21,22,23}. This analysis suggests that the overexpression of the candidate miRNA results in the downregulation of a common set of 125 genes mostly enriched for cell cycle-related processes. The novel miRNAs have distinct downstream candidate targets which are involved in cell cycle.

To characterise the mechanism of action of each candidate miRNA, we aimed to identify their direct targets in IL-1A/PDGF-BB-stimulated proliferating HSVSMCs through a bioinformatics pipeline developed for predicting functional miRNA-mRNA interactions for each candidate miRNA. We used multimiR²⁴, a prediction tool package that compiles several target prediction algorithms to obtain a comprehensive list of predicted targets per candidate novel miRNA. After those lists of predicted targets were created, we filtered them for genes that are significantly down-regulated following overexpression of each miRNA individually, compared to the miR-CTRL condition (FIG. 5A).

This led to the identification of candidate targets ranging from 3 to 173 in number per each of the individual candidate miRNAs (FIG. 5B), with a total of 493 candidate targets if sum the total targets of all 7 miRNAs are summed together. These candidate targets were characterised by both a likelihood of an existing miRNA-3′UTR interaction, as well as a resulting downregulation of gene expression. 93.5% of the identified 493 candidate targets are unique to one specific miRNA, while only 32 genes (6.5%) were candidate targets for 2 or more miRNAs (FIG. 5C). The genes most frequently targeted by the candidate miRNAs are IGF2BP3 and TIMP3 (targeted by 4 miRNAs each). The large number of individual targets and lack of complete overlap in candidate targets suggests that each miRNA has a distinct mechanism of action.

For miRNAs with more than 20 candidate targets (i.e. miR323-3p, miR491-3p, miR-449b-5p and miR892b), the Gene Ontology terms associated with these genes we assessed (FIG. 11) in order to understand if a specific miRNA has a critical role in a specific biological process by targeting several genes from this process. It was shown that miR-323a-3p targets are solely associated with “Cell Cycle”-related significantly enriched GO terms, revealing that miR-323-3p is a putative regulator of multiple cell cycle-related genes downstream. For miR-892b, we identified “spindle microtubules to kinetochore”-related enriched GO terms, a process associated to cell division, suggesting that the anti-proliferative phenotype induced by miR-892b in HSVSMCs can be linked to a downstream effect on the spindle apparatus. Other significantly enriched GO terms found for miR-323-3p, miR-892b and the other two candidate miRNAs with more than 20 candidate targets were “response to glucose”, “locomotion”, “response to calcium”and “lipid transportation”.

We next assessed whether each candidate miRNA could target genes in either of: i. the set of 125 commonly-downregulated genes identified above through differential expression analysis (FIG. 6A), or ii. any genes annotated under “Cell cycle” in GO terms. miR-323-3p, miR-449b-5p and miR-892b are capable of directly targeting candidates from the 125 common down-regulated genes, as visualised via differently shaped boxes in FIG. 5D. Next, we sought to reveal whether any of the 7 miRNA candidate targets could be involved in cell cycle process, progression and/or regulation using the GO term annotation “Cell Cycle”. It was found that every miRNA has at least one candidate target related to “Cell Cycle”(FIG. 5D).

Collectively so far, this data reveals the potential pathways and biological processes affected by mRNA inhibition following overexpression of our 7-novel miRNA in HSVSMCs. All 7 miRNAs induced transcriptomic changes due to the targeting of minimally overlapping sets of candidate genes, suggesting distinct mechanisms of action downstream between the miRNAs. Gross transcriptomic changes that result following overexpression of each of the novel miRNAs reveal the convergence towards affecting a core cell cycle-related gene network.

### Overexpression of Individual MiRNA Candidates Differentially Regulates HSVSMCS and HSVECs.

Preservation of the function of the endothelium and testing whether interventions are likely to interfere with HSVEC healing is key; therefore, we were interested in whether our miRNA candidates can cause phenotypic modulation in HSVEC. To test the effect of the miRNA candidates on the proliferation of HSVECs, we induced quiescence for 12 hours, transfected with miRNA mimics, and subsequently stimulated with FBS for 48 hours (FIG. 6A). Flow cytometric quantification of EdU incorporation revealed non-significant changes in proliferation after transfection with miR-1827, miR-323a-3p and miR-5681b, compared to miR-CTRL (FIG. 6B). By looking at the regulation of the cell cycle process-enriched set of 125 commonly downregulated genes from FIG. 4A at an RNA-sequencing dataset of HSVECs stimulated with FBS and transfected with either miR-CTRL or mimics for the candidate miRNA (according to the schematic in FIG. 6A), we observed that whilst in HSVSMCs there is clear differential regulation of those genes between the miR-CTRL condition and miRNA mimic-treatment, in HSVECs there is no distinctive pattern and high variability at the level of the transcriptome (FIG. 6C).

Overexpression of miR-892b, miR-491-3p, miR-449b-5p and miR-4774-3p Can Also Significantly Reduce PSVSMC Proliferation in Vitro

Testing the effect of the novel miRNA candidates in pig saphenous vein smooth muscle cell (PSVSMC) proliferation can provide essential information on the potential efficacy of the use of a large mammal model of vascular injury, such as the pig. PSVSMCs were quiesced for 48 h in basal medium, transfected, and stimulated with FBS (FIG. 7A). Candidates miR-892b, miR-491-3p, miR-449b-5p and miR-4774-3p significantly reduced FBS-induced proliferation of PSVSMCs (FIG. 7B). Out of our seven miRNA candidates, only miR-323a-3p is annotated in the pig genome. Using the UCSC genome browser²⁶, highly similar sequences to human pre-and mature miR-491-3p and miR-449b-5p in the pig genome were identified, although there is no miRbase annotation to suggest that these two mature miRNAs could be expressed and functional in PSVSMCs. Therefore, these two miRNAs are likely conserved. However, as shown by the significant effect of miR-892b on PSVSMC proliferation, conservation is not required for significant phenotypic effect.

These results indicate that all 7 novel candidate miRNAs are also capable of reducing unfavourable disease-inducing phenotypes in additional vascular cell types associated with the vein graft setting, making them of further therapeutic and translational interest.

### MiRNA-Overexpression Strategies for Treatment of Saphenous Vein Grafts Ex Vivo

For evaluating the effect of candidate miRNA overexpression, a reproducible model of saphenous vein injury and culture is used, associated with increasing VSMC proliferation and migration^{27,28}. Vein segments are exposed to miRNA mimics for 30 minutes, before stretching and culture for 7 days. Following treatment (as well as for our untreated controls), the tissue is collected and either: i) lysed for RNA isolation and quantification of candidate miRNA expression with qPCR, or ii) fixed for subsequent staining for the VSMC marker myosin heavy chain 11 (Myh11), the proliferation marker PCNA, as well as for the mature miRNA candidate using miRNA in situ hybridisation (FIG. 8A). To provide a proof of principle for the reduction of VSMC proliferation in this system following overexpression of candidate miRNA, we are focusing on miR-892b as it has the desired effects in HSVSMCs, HSVEC and PSVSMCs, simultaneously. However, the other miRNA candidates remain interesting as therapeutics, whose efficacy will be pursued to evaluate further. In addition, an adenoviral vector (Ad5) is to be tested for candidate miR-892b (FIG. 8B-C) for both validation of overexpression of the miRNA following transduction with the virus in HSVSMCs, as well as for validation of the anti-proliferative phenotype of the miRNA in these cells following adenoviral vector-mediated overexpression. Both these approaches will enable us to select the most efficient overexpression strategy for the candidate miRNA in saphenous vein segments ex vivo.

### Discussion

Aberrantly proliferative VSMCs are central players in the development of pathological vascular remodelling following stimuli that disrupt the integrity of the vessel wall. In saphenous vein graft failure, trauma in the vessel caused by surgical handling, as well as by haemodynamic stresses post implantation, results in the activation of resident quiescent VSMCs, characterised by increased rates of proliferation and migration. Here, we aimed to identify novel miRNA therapeutics based on their ability to reduce proliferation of VSMCs. By filtering for the most anti-proliferative miRNA candidates, that were also characterised by novelty in the literature, we generated a list of 7 miRNA candidates, and evaluated their functional role as inhibitors of cell proliferation and migration, without simultaneously inducing apoptosis nor senescence. For further analysis and evaluation of their potential therapeutic efficacy, we used an ex vivo model of saphenous vein graft injury. This study has successfully identified potential therapeutic candidates, which not only reduce pathological VSMC phenotypes associated with vascular remodelling, but also do not significantly affect HSVEC proliferation or significantly reduce PSVSMC proliferation. This renders them attractive potential therapeutics worth pursuing further studies with, in the context of saphenous vein injury and organ culture ex vivo, as well as injury-induced neointimal hyperplasia in vivo later on.

For the first time, a systematic and unbiased approach was used for the identification of therapeutic miRNA candidates, specifically in a vascular remodelling context, through a high-throughput, functional miRNA mimic screen. The miRNA library used contains mimics corresponding to the entirety of the annotated human mature miRNA sequences in miRbase version 21. Therefore, our choice of candidates was not confined by the already pre-existing miRNA literature and led to the identification of novel potential miRNA therapeutics. The miRNA sequences overexpressed in our screen included individual annotated −3 p and −5 p miRNA strands from all known miRNA loci, based on evidence that miRNA strands originating from the same miRNA locus could have different, or even opposing effects. We hypothesised that, due to −3 p and −5 p sequences carrying different seed sequences, their downstream mRNA targets, and subsequent effect on VSMC phenotype can possibly also differ²⁹.

Early-passage VSMCs originating from patients with pulmonary arterial hypertension and, therefore, characterised by high basal proliferation rates were used for the miRNA screen due to our goal being to specifically identify miRNAs capable of reducing VSMC proliferation linked to a state of pathological vascular injury. Following the initial miRNA screening step, we performed a round of validation using a panel of different cell types to increase confidence in the outcome of the miRNA screen. For further downstream evaluation of the miRNA candidates, we used in vitro, and currently use ex vivo models of saphenous vein graft injury.

Through RNA-sequencing, we were able to examine the changes induced by overexpression of individual miRNAs in the transcriptomic level.

Due to the strong anti-proliferative phenotype that we observed in HSVSMCs with miRNA overexpression in vitro, we were interested in whether the candidate miRNAs result in downstream regulation of cell cycle-related processes at the level of the transcriptome. We identified a strong convergence towards the regulation of the cell cycle via inducing downregulation of a core network of 125 genes. However, this common overarching effect on the transcriptome appears to be induced via different means per miRNA, as demonstrated through their distinct candidate target genes.

Following the identification of these candidate miRNAs, profiling of the transcriptomic changes that they induce via RNA-sequencing, and assessment of their function across different types of SMC and vascular cells, we performed further work to assess their effect in vitro, using HSVECs and PSVSMCs, in silico, using RNA-sequencing of HSVECs, as well as human vascular tissue. All miRNAs either induced significant reduction in PSVSMC proliferation and/or did not significantly affect HSVEC proliferation. These results interestingly demonstrate, respectively, a feasibility in using a pig model of vein graft failure to test miRNA candidates, as well as a HSVSMC-specific anti-proliferative effect exerted by some of the miRNAs which could interfere with HSVEC healing and compromise endothelial layer preservation. Candidate miR-892b was picked only to provide a proof of concept of ex vivo miRNA delivery and in situ reduction of VSMC proliferation in human primary tissue based on having no significant inhibitory effect on HSVEC proliferation, while at the same time significantly reducing PSVSMC proliferation. However, from a translational interest standpoint, all miRNAs remain interesting candidates, worthy of further evaluation.

Although VSMC proliferation can contribute to the important process of adaptive vascular remodelling following vascular injury, it can also result in intimal thickening, subsequent vessel occlusion and constriction of blood flow³⁰. The specific molecular mechanisms underlying the potential differences between beneficial and aberrant VSMC proliferation induced by injury remain unexplored. Consequently, for the purpose of this study, we are assessing the effect of the miRNAs on the total amount of VSMC proliferation that was observed in our HSV ex vivo model. Further studies in this context are required to provide a novel framework in targeting specifically of aberrantly proliferating VSMCs present the injured vessel wall.

### REFERENCES

1. Bochaton-Piallat MI, Bäck M. Novel Concepts For The Role Of Smooth Muscle Cells In Vascular Disease: Towards A New Smooth Muscle Cell Classification. Cardiovasc Res. 2018; 114:477-480.
2. Patel Mi, Ghosh P, Melrose J, Appleberg M. Smooth Muscle Cell Migration And Proliferation Is Enhanced In Abdominal Aortic Aneurysms. Aust N Z J Surg. 1996; 66:305-308.
3. Martin R. Bennett, Sanjay Sinha And Gary K. Owens; Vascular Smooth Muscle Cells In Atherosclerosis
4. Ballantyne Md, Pinel K, Dakin R, Vesey At, Diver L, Mackenzie R, Garcia R, Welsh P, Sattar N, Hamilton G, Et Al. Smooth Muscle Enriched Long Noncoding Rna (Smilr) Regulates Cell Proliferation. Circulation. 2016; 133:2050-2065. Doi: 10.1161/Circulationaha. 115.021019
5. Roopmani, P., Sethuraman, S., Satheesh, S. and Maheswari Krishnan, U., 2016. The metamorphosis of vascular stents: passive structures to smart devices. RSC Advances, 6(4), pp. 2835-2853.
6. PREVENT IV Investigators*, 2005. Efficacy and Safety of Edifoligide, an E2F Transcription Factor Decoy, for Prevention of Vein Graft Failure Following Coronary Artery Bypass Graft Surgery. JAMA, 294(19), p. 2446.
7. Bartel Dp. Micrornas: Genomics, Biogenesis, Mechanism, And Function. Cell. 2004 Jan. 23; 116(2): 281-97. Doi: 10.1016/S0092-8674(04)00045-5. Pmid: 14744438
8. Lu Y, Thavarajah T, Gu W, Cai J, Xu Q. Impact Of Mirna In Atherosclerosis. Arterioscler Thromb Vasc Biol. 2018 September; 38(9): E159-E170. Doi: 10.1161/Atvbaha. 118.310227. Pmid: 30354259; Pmcid: Pmc6795547
9. Vacante F, Denby L, Sluimer JC, Baker AH. The function of miR-143, miR-145 and the MiR-143 host gene in cardiovascular development and disease. Vascul Pharmacol. 2019 January; 112:24-30. doi: 10.1016/j. vph.2018.11.006. Epub 2018 Nov. 29. PMID: 30502421; PMCID: PMC6395947.
10. Vacante, F., Rodor, J., Lalwani, M., Mahmoud, A., Bennett, M., De Pace, A., Miller, E., Van Kuijk, K., de Bruijn, J., Gijbels, M., Williams, T., Clark, M., Scanlon, J., Doran, A., Montgomery, R., Newby, D., Giacca, M., O'Carroll, D., Hadoke, P., Denby, L., Sluimer, J. and Baker, A., 2021. CARMN Loss Regulates Smooth Muscle Cells and Accelerates Atherosclerosis in Mice. Circulation Research, 128(9), pp. 1258-1275.
11. Rupaimoole, R., Slack, F. Microrna Therapeutics: Towards A New Era For The Management Of Cancer And Other Diseases. Nat Rev Drug Discov 16, 203-222 (2017). Https://Doi. Org/10.1038/Nrd.2016.246
12. Gabisonia, K., Prosdocimo, G., Aquaro, G.D. Et Al. Microrna Therapy Stimulates Uncontrolled Cardiac Repair After Myocardial Infarction In Pigs. Nature 569, 418-422 (2019). Https://Doi.Org/10.1038/S41586-019-1191-6
13. Qin, S. and Zhang, C., 2011. MicroRNAs in Vascular Disease. Journal of Cardiovascular Pharmacology, 57(1), pp. 8-12.
14. Wan, S., George, S., Berry, C. Et Al. Vein Graft Failure: Current Clinical Practice And Potential For Gene Therapeutics. Gene Ther 19, 630-636 (2012). Https://Doi.Org/10.1038/Gt.2012.29
15. Kim Sc, Rho Mc, Lee Hs, Kim Yk, Kim K. Caspase-3-Dependent Apoptosis In Vascular Smooth Muscle Cell By Proteasome Inhibition. J Cardiovasc Pharmacol. 2003 October; 42(4): 554-60. Doi: 10.1097/00005344-200310000-00014. Pmid: 14508242.
16. Zhang Y, Liang Q, Zhang Y, Hong L, Lei D, Zhang L. Olmesartan Alleviates Bleomycin-Mediated Vascular Smooth Muscle Cell Senescence Via The Mir-665/Sdc1 Axis. Am J Transl Res. 2020; 12(9): 5205-5220. Published 2020 Sep. 15
17. Love, M.I., Huber, W. & Anders, S. Moderated Estimation Of Fold Change And Dispersion For Rna-Seq Data With Deseq2. Genome Biol 15, 550 (2014).
18. Ashburner et al. Gene ontology: tool for the unification of biology. Nat Genet. May 2000; 25(1): 25-9.
19. The Gene Ontology resource: enriching a GOId mine. Nucleic Acids Res. January 2021; 49(D1): D325-D334.
20. Alexa A, Rahnenfuhrer J (2022). topGO: Enrichment Analysis for Gene Ontology. R package version 2.48.0.
21. Kanehisa, M. and Goto, S. ; KEGG: Kyoto Encyclopedia of Genes and Genomes. Nucleic Acids Res. 28, 27-30 (2000).
22. Kanehisa, M; Toward understanding the origin and evolution of cellular organisms. Protein Sci. 28, 1947-1951 (2019)
23. Kanehisa, M., Furumichi, M., Sato, Y., Ishiguro-Watanabe, M., and Tanabe, M. ; KEGG: integrating viruses and cellular organisms. Nucleic Acids Res. 49, D545-D551 (2021).
24. Ru Y, Kechris Kj, Tabakoff B, Et Al. The MultimiR R Package And Database: Integration Of Microrna-Target Interactions Along With Their Disease And Drug Associations. Nucleic Acids Res. 2014; 42(17): E133. Doi: 10.1093/Nar/Gku631
25. Jensen L J, Kuhn M, Stark M, Chaffron S, Creevey C, Muller J, Doerks T, Julien P, Roth A, Simonovic M, Bork P, von Mering C. STRING 8—a global view on proteins and their functional interactions in 630 organisms. Nucleic Acids Res. 2009 January; 37(Database issue): D412-6. doi: 10.1093/nar/gkn760. Epub 2008 Oct. 21. PMID: 18940858; PMCID: PMC2686466.
26. Kent Wj, Sugnet Cw, Furey Ts, Roskin Km, Pringle Th, Zahler Am, Haussler D. The Human Genome Browser At UCSC. Genome Res. 2002 June; 12(6): 996-1006. Doi: 10.1101/Gr.229102. Pmid: 12045153; Pmcid: Pmc186604.
27. Soyombo AA, Angelini GD, Bryan AJ, Jasani B, Newby AC. Intimal proliferation in an organ culture of human saphenous vein. Am J Pathol. 1990; 137:1401-1410.
28. Angelini GD, Soyombo AA, Newby AC. Winner of the ESVS prize 1990. Smooth muscle cell proliferation in response to injury in an organ culture of human saphenous vein. Eur J Vasc Surg. 1991; 5:5-12.
29. Zhang, Z., Pi, J., Zou, D. 2019. Microrna Arm-Imbalance In Part From Complementary Targets Mediated Decay Promotes Gastric Cancer Progression. Nat Commun 10, 4397
30. Owens, C.D., Wake, N., Jacot, J.G., Gerhard-Herman, M., Gaccione, P., Belkin, M., Creager, M.A. & Conte, M.S. 2006. Early Biomechanical Changes In Lower Extremity Vein Grafts-Distinct Temporal Phases Of Remodeling And Wall Stiffness. Journal Of Vascular Surgery, 44, 740-746.

1. A method of:
(i) modulating VSMC proliferation;
(ii) treating or preventing diseases or conditions characterised by VSMC proliferation;
(iii) treating or preventing vein graft failure/late vein graft failure;
(iv) treating or preventing vein graft failure/late vein graft failure following surgery;
(v) treating or preventing vein graft failure/late vein graft failure following coronary bypass surgery, including, for example using saphenous veins;
(vi) treating or preventing atherosclerosis;
(vii) treating, preventing or modulating vascular remodelling;
(viii) treating or preventing vascular injury; and/or
(ix) treating or preventing vascular injury where VSMC proliferation is an important phenotype;
said method comprising administering a miR modulator to a subject in need thereof.

2. The method of claim 1, wherein diseases or conditions characterised by VSMC proliferation include one or more selected from:
(i) in stent restenosis;
(ii) arteriovenous fistulas;
(iii) intimal thickening (hyperplasia);
(iv) vessel occlusion; and/or
(v) complications arising from constricted/restricted blood flow.

3. The method of claim 1, wherein the vascular remodelling is aberrant.

4. The method of claim 1, wherein the method is for the treatment or prevention of:
(i) atherosclerosis;
(ii) pulmonary hypertension;
(iii) aortic aneurism; and/or
(iv) intimal hyperplasia underlying saphenous vein graft failure.

5. The method of claim 1, wherein the miR modulator increases the expression of one or more of the following miR(s):
(i) miR-892b;
(ii) miR-1827;
(iii) miR-332a-3p;
(iv) miR-449b-5p;
(v) miR-491-3p;
(vi) miR-4774-3p; and/or
(vii) miR-5681b.

6. The method of claim 1, wherein the miR modulator increases the expression of one or more of the following miR(s):
(i) miR-892b;
(ii) miR-1827;
(iii) miR-332a-3p; and/or
(iv) miR-5681b.

7. The method of claim 1, wherein the miR modulator increases the expression of miR-892b.

8-11. (canceled)

12. The method of claim 1, wherein the miR modulator comprises, consists essentially of or consists of a miR mimic.

13. The method of claim 1, wherein the miR modulator is provided in the form of a vector for delivery to a cell.

14. The method of claim 13, wherein the vector is a viral vector.

15. The method of claim 1, wherein the miR modulator reduces aberrant proliferation and/or migration of VSMCs.

16. The method of claim 1, wherein the miR modulator is administered directly to a vessel wall to be treated.

17. The method of claim 16, wherein the vessel wall is selected from the group consisting of:
(i) a vessel that has been repaired through surgery; or
(ii) a vessel which shows signs of disease and/or of injury or damage; and
(iii) a saphenous vein wall.

18. The method of claim 16, wherein the miR modulator is administered directly to a vessel wall within the clinical window during CABG when the SVG is available between harvesting and implantation.

19. The method of claim 1, wherein the subject is selected from the group consisting of:
(i) a subject suffering from a disease or condition characterised by VSMC proliferation;
(ii) a subject suffering from vein graft failure/late vein graft failure;
(iii) a subject susceptible or predisposed to a disease or condition characterised by VSMC proliferation;
(iv) a subject susceptible or predisposed to vein graft failure/late vein graft failure;
(v) a subject undergoing or convalescing from coronary bypass surgery;
(vi) a subject undergoing or convalescing from coronary bypass surgery using saphenous veins;
(vii) a subject suffering from atherosclerosis;
(viii) a subject predisposed/susceptible to atherosclerosis;
(ix) a subject suffering from vascular injury; and
(x) a subject predisposed or susceptible to vascular injury, including vascular injury where VSMC proliferation is an important phenotype.
